(19) United States
(12) Patent Application Publication
(10) Pub. No.: US 2026/0232750 A1
(43) Pub. Date: Aug. 13, 2026

(54) COMPOSITION AND METHOD FOR INHIBITING ADVANCED GLYCATION END PRODUCTS

(71) Applicant: ACCESS BUSINESS GROUP INTERNATIONAL LLC, Ada, MI (US)

(72) Inventors: Huiji ZHOU, Shanghai (CN); Bo LI, Shanghai (CN); Tingzhao LI, Shanghai (CN); Feijie WANG, Shanghai (CN); Xiuyu JIANG, Shanghai (CN)

(21) Appl. No.: 18/859,515

(22) PCT Filed: Apr. 24, 2023
(86) PCT No.: PCT/US2023/019609

§ 371 Date: Oct. 23, 2024

(30) Foreign Application Priority Data
Apr. 25, 2022 (CN) 202210437608.5

(51) Int. Cl. A61K 36/8984; A61K 8/9789; A61K 8/9794; A61K 36/46; A61K 36/47; A61K 36/489; A61K 36/73; A61K 36/808; A61P 19/00; A61P 39/06; A61P 43/00; A61Q 19/08

(52) U.S. Cl. CPC A61K 36/8984; A61K 8/9789; A61K 8/9794; A61K 36/46; A61K 36/47; A61K 36/489; A61K 36/73; A61K 36/808; A61P 19/00; A61P 39/06; A61P 43/00; A61Q 19/08; A61K 2236/331; A61K 2800/522; A61K 2800/92

(57) ABSTRACT

A composition for administration to a subject is disclosed. The composition comprises at least one botanical active component. The botanical active component is present in the composition in an amount effective to inhibit advanced glycation end products (AGEs) in skin of the subject. The botanical active component comprises at least one extract selected from the group consisting of: i) an extract of Dendrobium officinale flower; ii) an extract of Scrophularia ningpoensis; iii) an extract of Eucommia ulmoides; and iv) combinations of i) to iii). The composition may be in the form of a topical composition, e.g., for topical application. The composition may also be in the form of an oral composition, e.g., for ingestion. A method of inhibiting advanced glycation end products (AGEs) in skin of a subject is also provided. The method comprises administering an effective amount of the composition to the subject.

### CROSS-REFERENCE TO RELATED APPLICATIONS

This application claims priority to and all benefits of Chinese Patent Application No. 202210437608.5 filed on 25 Apr. 2022, the disclosure of which is incorporated herein by reference.

### FIELD OF THE INVENTION

The present invention relates generally to compositions and methods for inhibiting protein glycation and, more specifically, to a botanical extract-containing composition for inhibiting advanced glycation end products (AGEs) in skin of a subject. Related methods are also provided.

### BACKGROUND OF THE INVENTION

Advanced glycation end-products (AGEs) are the final products of a series of chemical reactions initiated by the attachment of reducing sugars to free amino groups in proteins, lipids and nucleic acids. AGEs are commonly viewed as a group of complex and heterogeneous compounds. Recently, their accumulation in vivo has been implicated as a major pathogenic process in diabetic complications, including neuropathy, nephropathy, retinopathy and cataract and other health disorders, such as atherosclerosis, Alzheimer's disease, etiology of ovarian aging, polycystic ovary syndrome (PCOS) infertility and normal aging. Meanwhile, some highly reactive carbonyl species (RCS) formed during the formation of AGEs are also believed to play an important role in diabetic complications.

In view of the foregoing, there remains an opportunity to provide new and useful advanced glycation end-product (AGE) inhibitors. Such AGE inhibitors can offer potential preventive and therapeutic approaches for lowering the risks of diabetic or other pathogenic complications caused by AGE formation. As such, there also remains an opportunity to provide new and useful compositions and methods for inhibiting AGEs.

### BRIEF SUMMARY OF THE INVENTION

A composition for administration to a subject is provided. The composition comprises at least one botanical active component. The botanical active component is generally present in the composition in an amount effective to inhibit advanced glycation end products (AGEs) in skin of the subject. The botanical active component comprises at least one extract selected from the group consisting of: i) an extract of Dendrobium officinale flower; ii) an extract of Scrophularia ningpoensis; iii) an extract of Eucommia ulmoides; and iv) combinations of i) to iii).

In various embodiments, the composition is a topical composition that is formulated for topical administration to the subject. In other embodiments, the composition is an oral composition that is formulated for oral administration to the subject.

The composition can be used for inhibiting AGEs in skin of a subject. A method of inhibiting AGEs in skin of a subject comprises administering an effective amount of the composition to the subject.

### BRIEF DESCRIPTION OF THE DRAWINGS

FIG. 1 is a plot showing the average inhibition rate of fluorescent advanced glycation end-product (AGE) using Rutin as a control;

FIG. 2 is a plot showing the average inhibition rate of fluorescent AGE using Dendrobium officinale (flower) (“DOF”)-aqueous extract;

FIG. 3 is a plot showing the average inhibition rate of fluorescent AGE using DOF-ethanol extract;

FIG. 4 is a plot showing the average inhibition rate of fluorescent AGE using Scrophulariae ningpoensis (radix)-aqueous extract;

FIG. 5 is a plot showing the average inhibition rate of fluorescent AGE using Eucommia ulmoides (male flowers)-aqueous extract.

FIG. 6 is a chart showing the effect of DOF-aqueous extract on human dermal fibroblasts (HDFs);

FIG. 7 includes images of the anti-glycation effect of DOF-aqueous extract on HDFs;

FIG. 8 is a flow chart of online detection of antioxidants;

FIG. 9 are charts showing antioxidant potency of DOF extracts;

FIG. 10 is a chart showing COX-2 inhibition of DOF hot-water extract (“DOF-W”);

FIG. 11 are charts showing anti-glycation activity of DOF-W;

FIG. 12 includes images showing effects of DOF extract on the expression of collagens by immunofluorescent (IF) staining;

FIG. 13 includes images of fibroblasts with SA-3-gal staining; and

FIG. 14 is the total ion chromatogram of an aqueous extract of a sample under both negative and positive ion mode.

### DETAILED DESCRIPTION OF THE CURRENT EMBODIMENTS

A composition for administration to a subject is disclosed. The composition comprises at least one botanical active component. The composition is described below, followed by description of associated uses and methods.

As will be understood in view of this disclosure, the composition is not particularly limited aside from the botanical active component and, in particular, the extract(s) thereof, as well as the related components and methods. As such, the composition may be formulated, for example, as a topical composition (e.g., a cosmetic composition), or as an oral composition or as a nutraceutical, pharmaceutical, or supplement, and may be utilized as a unique and stand-alone therapeutic or in combination with other therapeutics compatible therewith.

The composition is useful for treating, preventing, and/or ameliorating various conditions, such as those associated with aging. Specifically, as will be appreciated in view of the description and examples below, the composition of the present embodiments is believed to be capable of inhibiting protein glycation and, more specifically, inhibiting advanced glycation end products (AGEs) in skin of a subject.

As such, the composition may be utilized to treat (i.e., slow, prevent, reverse, etc.) age-related or other conditions generally associated with AGEs, such as diabetic complications, including neuropathy, nephropathy, retinopathy and cataract and other health disorders, such as atherosclerosis, Alzheimer's disease, etiology of ovarian aging, polycystic ovary syndrome (PCOS) infertility and normal aging.

As introduced above, the composition comprises a botanical active component. More specifically, the botanical active component comprises, optionally consists essentially of, or optionally consists of at least one extract selected from the group consisting of: i) an extract of Dendrobium officinale flower; ii) an extract of Scrophularia ningpoensis; iii) an extract of Eucommia ulmoides; and iv) combinations of i) to iii).

In certain embodiments, only one of the three extracts i) to iii) is present in the composition. In other embodiments, only two of the three extracts i) to iii) is present in the composition. In yet other embodiments, all three of the extracts i) to iii) are present in the composition. Each of the individual extracts i) to iii) may be referred to simply as the “botanical extract” or collectively as the “botanical extracts” and are described in turn below.

The term “extract” is used herein in the conventional sense to refer to a composition that has been obtained via fluid extraction from a source material. As such, the term “botanical extract” is to be understood as a composition obtained via fluid extraction (e.g., solvent extraction, gas extraction, CO₂ extraction, etc.) from a botanical source (i.e., a plant material). Botanical extracts suitable for use in the composition can be obtained via any extraction method, or combination of such methods, known in the art, including water extractions, steam extractions, solvent extractions, etc. Exemplary extraction techniques are described below. However, the botanical extracts are generally not limited to a particular extraction method, or additional/adjuvant techniques used to obtain the botanical extracts, but rather may vary according to the parameters described herein. Additionally, an extraction step is not required to prepare the botanical active component and/or the composition, as suitable extracts (e.g. standardized extracts) are readily available from a number of commercial suppliers.

Botanical extracts suitable for use in, or as, the botanical active component include those obtained via solvent extraction, e.g. via use of a polar solvent such as an alcohol (e.g. methanol, ethanol, butylene glycol, etc.), ether (e.g. diethyl ether, methyl tert butyl ether, etc.), ketone (e.g. acetone), ester (e.g. ethyl acetate), phenol, water, and the like, a nonpolar solvent such as benzene, xylenes, toluene, etc., as well as derivatives, modifications, and combinations thereof (e.g. solvent-water blends, including alcohol-water, acetone-water, etc.). Additional and alternative extraction techniques include sequential fractionations, total hydro-ethanolic extractions, lump-sum extractions, supercritical fluid extractions (e.g. with CO₂), and the like, as well as those utilizing sequential or secondary extractions from a first extract (e.g. a non-polar solvent extract of a botanical extract obtained from a polar solvent extraction) or other processing techniques such as filtration, purification, distillation, dehydration, evaporation, concentration, drying, etc. Specific examples of suitable extraction methods are described in U.S. Pat. No. 7,897,184, which is incorporated by reference herein.

As understood in the art, various sections or parts of plants can be used to obtain the essential oils and extracts, such as bark, berries, flowers, fruits, leaves, peels, resins, rhizomes, roots, seeds, and/or woods. Essential oils can be obtained by a number of processes, such as by distillation (e.g. using steam), expression, solvent extraction, absolute oil extraction, resin tapping, and/or cold pressing.

In various embodiments, the solvent used to obtain suitable botanical extracts for this disclosure is one in which the resulting botanical extract and/or a subsequent form thereof (e.g. botanical extract powder) is suitable for ingestion. For example, the solvent is water or ethanol.

In one example, the botanical extracts can be obtained using an organic solvent extraction technique. In another example, solvent sequential fractionation can be used to obtain the botanical extracts. Total hydro-ethanolic extraction techniques can also be used to obtain the botanical extracts. Generally, this is referred to as a lump-sum extraction. The botanical extract generated in the process will contain a broad variety of phytochemicals present in the extracted material including fat and water-soluble phytochemicals. Following collection of the botanical extract solution, the solvent will be evaporated, resulting in the botanical extract.

Total ethanol extraction may also be used. This technique uses ethanol as the solvent. This extraction technique generates a botanical extract that may include fat soluble and/or lipophilic compounds in addition to water-soluble compounds. Total methanol extraction may also be used in a similar manner with similar results.

Another example of an extraction technique that can be used to obtain the botanical extracts is supercritical fluid carbon dioxide extraction (SFE). In this extraction procedure, the material to be extracted is not exposed to any organic solvents. Rather, the extraction solvent is carbon dioxide (CO₂), with or without a modifier, in super-critical conditions (e.g. >31.3° C. and >73.8 bar). Those of skill in the art will appreciate that temperature and pressure conditions can be varied to obtain the best yield of botanical extract. This technique generates a botanical extract of fat soluble and/or lipophilic compounds, similar to total hexane and ethyl acetate extraction techniques, which may also be used.

Each of the extraction methods above also may include and/or be utilized in combination with one or more additional processing steps understood in the art. For example, plant material may be comminuted, smashed, ground, etc. There also may be one or more filtration steps to remove, for example, cellulosic/fibrous or other solid materials. There also may be one or more purification steps to remove, for example, certain constituents and/or contaminants. Such purification may be accomplished, for example, by distillation, evaporation, centrifugation, etc. There also may be one or more concentration and/or drying steps to remove water and/or other volatiles, e.g. alcohol, lighter compounds, VOCs, etc. Moreover, acids and/or bases may be added to adjust pH or neutralize. Depending on the desired form of the final/end botanical extract, one can also utilize various additional steps understood in the art, such as screening, pressing, milling, grinding, mixing, dispersing, etc. It is to be appreciated that combinations of these additional processing steps in duplicative and/or different orders is also contemplated.

Dendrobium officinale

In some embodiments, the botanical active component, and thus the composition, comprises the extract of Dendrobium officinale flower, i.e., an extract comprising, optionally consisting essentially of material from the flowering plant species Dendrobium officinale. The Dendrobium officinale flower extract is not particularly limited, and may comprise or be any flower extract or combination of flower extracts from a Dendrobium officinale plant suitable for use in the embodiments herein. More specifically, exemplary Dendrobium officinale flower extracts include those capable of inhibiting AGEs or eliciting/exhibiting any other such activities described herein as part of the botanical active component.

Dendrobium officinale has been reported to contain various bioactive components, such as polysaccharides, bibenzyls, phenanthrenes, and flavonoids. Dendrobium officinale may simply be referred to as D. officinale, or via various other names such as Dendrobium officinale Kimura et. Migo, or “Tie Pi Shi Hu”.

At times, Dendrobium officinale may also be referred to as “Dendrobium candidum”. As further background, in the 2005 Chinese Pharmacopoeia (ChP), tiepishihu was one species of medicinal shihu called Dendrobium candidum Wall. ex Lindl.; however, this Latin name was later disputed and considered the synonym of Dendrobium moniliforme (L.) Sw. In the 2010 ChP, tiepishihu was renamed as Dendrobium officinale Kimura et Migo (a.k.a. Dendrobium officinale or D. officinale).

Specific examples of Dendrobium officinale flower extracts are known in art. As such, the Dendrobium officinale flower extract may be purchased or otherwise obtained commercially from various sources, prepared (e.g. using any conventional extraction technique(s) known in the art, such as any of those described herein), or combinations thereof. Specific examples of commercially available extracts include “Freeze Dried Dendrobium candidum Flower Extract Powder” from DAMIN INTERNATIONAL GROUP of Fujian, China; and Dendrobium officinale Kimura et. Migo Flower (water) Extract Powder from QINGYUN SHAN Pharmaceutical of Guangdong, China.

In certain embodiments, the extract of Dendrobium officinale flower is obtained by water extracting (or aqueous extracting) flowers or flower-based plant material of Dendrobium officinale. In further or other embodiments, the extract of Dendrobium officinale flower is obtained by alcohol extracting (e.g., ethanol extracting), flowers or flower-based plant material of Dendrobium officinale. The flowers may be fresh or dried, typically dried to prevent decay. The dried flowers may then be formed into a powder, which can be used as the extract itself, or more typically, the powdered flower is further processed to form the extract as like described below.

As will be understood by those of skill in the art, Dendrobium officinale is primarily cultivated for its flower. The flowers can be from one or more plants, and can be fresh, dried, or otherwise aged.

By way of example, certain extracts can be obtained where Dendrobium officinale flower is pulverized to a homogeneous size in a mill. Next, the resulting powder is extracted using a water or ethanol solution. The solution is then filtered and the filtrate can be concentrated under reduced pressure to yield a syrup. The syrup can then be freeze-dried to dryness to obtain extract.

In various embodiments, the botanical active component consists of the extract of Dendrobium officinale flower. In further or other embodiments, the composition is substantially to completely free of components obtained from non-flower-based plant material of Dendrobium officinale. In these embodiments, the non-flower-based plant material of Dendrobium officinale can be, for example, the root, bark, rhizome, leaf, or stem of a Dendrobium officinale plant. Without being bound by any particular theory, it is believed that the flower of Dendrobium officinale is most useful for anti-glycation effect; whereas other parts of Dendrobium officinale are not (as illustrated in the Examples section below).

Suitable flower extracts may be processed (e.g. defatted, partially defatted, ground, dried, precipitated, washed, filtered, mesh-sorted, extracted, distilled, concentrated, etc.) to obtain the Dendrobium officinale flower extract. Likewise, Dendrobium officinale flower may be extracted in raw form, or processed prior to extraction of the Dendrobium officinale flower extract (e.g. used in raw form, suspended form, dehydrated form, concentrated form, etc.).

The amount of the Dendrobium officinale extract utilized in the botanical active component may vary, and will be selected based on the number and types of components being utilized in the botanical active component. In certain embodiments, the botanical active component comprises from 1 to 2000 mg of the Dendrobium officinale extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Dendrobium officinale extract in an amount of at least 1, optionally of at least about 10, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Dendrobium officinale extract in an amount of ≤100, ≤250, ≤500, ≤750, ≤1000, ≤2000, ≤5000 mg. In various embodiments, the botanical active component can include an amount of Dendrobium officinale extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, ≤40, ≤50, ≤60, ≤70, ≤80, ≤90, and ≤99 wt. %, respectively, based on the total weight of the botanical active component.

In certain embodiments, the botanical active component comprises more than one Dendrobium officinale extract, such as 2, 3, 4, or more Dendrobium officinale extracts. In such embodiments, each Dendrobium officinale extract is independently selected, may be the same as or different from any other Dendrobium officinale extract, and each utilized in an amount as described above.

The Dendrobium officinale extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Dendrobium officinale extract(s) utilized. It will be appreciated that the Dendrobium officinale extract may be combined with the carrier vehicle, if utilized, prior to, during, or after being combined with any other components of the botanical active component and/or composition.

Scrophularia ningpoensis

In some embodiments, the botanical active component, and thus the composition, comprises the extract of Scrophularia ningpoensis, i.e., an extract comprising, optionally consisting essentially of material from the plant species Scrophularia ningpoensis. The Scrophularia ningpoensis extract is not particularly limited, and may comprise or be any extract or combination of extracts from a Scrophularia ningpoensis plant suitable for use in the embodiments herein. More specifically, exemplary Scrophularia ningpoensis extracts include those capable of inhibiting AGEs or eliciting/exhibiting any other such activities described herein as part of the botanical active component.

Scrophularia ningpoensis has been reported to contain various bioactive components, such as iridoids, phenolic glycosides, phenolic acids, alkaloids, flavonoids, triterpenes and other compounds. Scrophularia ningpoensis may simply be referred to as S. ningpoensis, or via various other names such as Scrophularia ningpoensis Hemsl., Ningpo figwort, Chinese figwort, “Xuan Shen” (or Radix Scrophulariae), “Hu Huang Lian”, “Shen Nong Ben Cao Jing” (or “Shen Nong's Herbal”), or “Yuan Shen”.

Specific examples of Scrophularia ningpoensis extracts are known in art. As such, the Scrophularia ningpoensis extract may be purchased or otherwise obtained commercially from various sources, prepared (e.g. using any conventional extraction technique(s) known in the art, such as any of those described herein), or combinations thereof. In certain embodiments, the extract of Scrophularia ningpoensis is obtained by water extracting (or aqueous extracting) plant material of Scrophularia ningpoensis. In further or other embodiments, the extract of Scrophularia ningpoensis is obtained by alcohol extracting (e.g., ethanol extracting), plant material of Scrophularia ningpoensis.

As will be understood by those of skill in the art, Scrophularia ningpoensis is primarily cultivated for its radix, rhizome, and/or roots. As such, in various embodiments, the extract of Scrophularia ningpoensis is an extract of Scrophularia ningpoensis radix. Suitable extractions include those noted above, e.g., water and ethanol extractions of roots. The roots can be from one or more plants, and can be fresh, dried, or otherwise aged.

By way of example, certain extracts can be obtained where Scrophularia ningpoensis (e.g. radix) is pulverized to a homogeneous size in a mill. Next, the resulting powder is extracted using a water or ethanol solution. The solution is then filtered and the filtrate can be concentrated under reduced pressure to yield a syrup. The syrup can then be freeze-dried to dryness to obtain extract.

The Scrophularia ningpoensis extract may comprise material from any part of the plant, or combinations of parts, and is not limited to root extracts. For example, the Scrophularia ningpoensis extract may comprise material extracted from one or more parts of a Scrophularia ningpoensis plant, including the root, stem, bark, rhizome, leaf, bud, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Scrophularia ningpoensis extract. Likewise, the Scrophularia ningpoensis plant may be extracted in raw form, or processed prior to extraction of the Scrophularia ningpoensis extract (e.g. used in raw form, suspended form, dehydrated form, concentrated form, etc.). In certain embodiments, the botanical active component comprises a Scrophularia ningpoensis extract comprising material obtained (i.e., extracted) from roots of Scrophularia ningpoensis.

The amount of the Scrophularia ningpoensis extract utilized in the botanical active component may vary, and will be selected based on the number and types of components being utilized in the botanical active component. In certain embodiments, the botanical active component comprises from 1 to 2000 mg of the Scrophularia ningpoensis extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Scrophularia ningpoensis extract in an amount of at least 1, optionally of at least about 10, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Scrophularia ningpoensis extract in an amount of ≤100, ≤250, ≤500, ≤750, ≤1000, ≤2000, ≤5000 mg. In various embodiments, the botanical active component can include an amount of Scrophularia ningpoensis extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, ≤40, ≤50, ≤60, ≤70, ≤80, ≤90, and ≤99 wt. %, respectively, based on the total weight of the botanical active component.

In certain embodiments, the botanical active component comprises more than one Scrophularia ningpoensis extract, such as 2, 3, 4, or more Scrophularia ningpoensis extracts. In such embodiments, each Scrophularia ningpoensis extract is independently selected, may be the same as or different from any other Scrophularia ningpoensis extract, and each utilized in an amount as described above.

The Scrophularia ningpoensis extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Scrophularia ningpoensis extract(s) utilized. It will be appreciated that the Scrophularia ningpoensis extract may be combined with the carrier vehicle, if utilized, prior to, during, or after being combined with any other components of the botanical active component and/or composition.

Eucommia ulmoides

In some embodiments, the botanical active component, and thus the composition, comprises the extract of Eucommia ulmoides, i.e., an extract comprising, optionally consisting essentially of material from the flowering plant species Eucommia ulmoides. The Eucommia ulmoides extract is not particularly limited, and may comprise or be any extract or combination of extracts from a Eucommia ulmoides plant suitable for use in the embodiments herein. More specifically, exemplary Eucommia ulmoides extracts include those capable of inhibiting AGEs or eliciting/exhibiting any other such activities described herein as part of the botanical active component.

Eucommia ulmoides has been reported to contain various bioactive components, such as lignans, iridoids (e.g., iridoid glucoside geniposidic acid), phenolics, steroids, terpenoids, and flavonoids. Eucommia ulmoides may simply be referred to as E. ulmoides, or via various other names such as Eucommia ulmoides Oliv., or dùzhòng.

Specific examples of Eucommia ulmoides extracts are known in art. As such, the Eucommia ulmoides extract may be purchased or otherwise obtained commercially from various sources, prepared (e.g. using any conventional extraction technique(s) known in the art, such as any of those described herein), or combinations thereof. In certain embodiments, the extract of Eucommia ulmoides is obtained by water extracting (or aqueous extracting) plant material of Eucommia ulmoides. In further or other embodiments, the extract of Eucommia ulmoides is obtained by alcohol extracting (e.g., ethanol extracting), plant material of Eucommia ulmoides.

As will be understood by those of skill in the art, Eucommia ulmoides is primarily cultivated for its flower, and more primarily for its male (or staminate) flower. As such, in various embodiments, the extract of Eucommia ulmoides is an extract of Eucommia ulmoides male flower. Suitable extractions include those noted above, e.g., water and ethanol extractions of male flowers. The male flowers can be from one or more plants, and can be fresh, dried, or otherwise aged.

By way of example, certain extracts can be obtained where Eucommia ulmoides (e.g. male flower) is pulverized to a homogeneous size in a mill. Next, the resulting powder is extracted using a water or ethanol solution. The solution is then filtered and the filtrate can be concentrated under reduced pressure to yield a syrup. The syrup can then be freeze-dried to dryness to obtain extract.

The Eucommia ulmoides extract may comprise material from any part of the plant, or combinations of parts, and is not limited to (male) flower extracts. For example, the Eucommia ulmoides extract may comprise material extracted from one or more parts of a Eucommia ulmoides plant, including the root, stem, bark, rhizome, leaf, bud, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Eucommia ulmoides extract. Likewise, the Eucommia ulmoides plant may be extracted in raw form, or processed prior to extraction of the Eucommia ulmoides extract (e.g. used in raw form, suspended form, dehydrated form, concentrated form, etc.). In certain embodiments, the botanical active component comprises a Eucommia ulmoides extract comprising material obtained (i.e., extracted) from male flowers of Eucommia ulmoides.

The amount of the Eucommia ulmoides extract utilized in the botanical active component may vary, and will be selected based on the number and types of components being utilized in the botanical active component. In certain embodiments, the botanical active component comprises from 1 to 2000 mg of the Eucommia ulmoides extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Eucommia ulmoides extract in an amount of at least 1, optionally of at least about 10, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Eucommia ulmoides extract in an amount of ≤100, ≤250, ≤500, ≤750, ≤1000, ≤2000, ≤5000 mg.

In various embodiments, the botanical active component can include an amount of Eucommia ulmoides extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, ≤40, ≤50, ≤60, ≤70, ≤80, ≤90, and ≤99 wt. %, respectively, based on the total weight of the botanical active component.

In certain embodiments, the botanical active component comprises more than one Eucommia ulmoides extract, such as 2, 3, 4, or more Eucommia ulmoides extracts. In such embodiments, each Eucommia ulmoides extract is independently selected, may be the same as or different from any other Eucommia ulmoides extract, and each utilized in an amount as described above.

The Eucommia ulmoides extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Eucommia ulmoides extract(s) utilized. It will be appreciated that the Eucommia ulmoides extract may be combined with the carrier vehicle, if utilized, prior to, during, or after being combined with any other components of the botanical active component and/or composition.

### Supplemental Extracts

In various embodiments, the composition further comprises one or more supplemental extracts. More specifically, in addition to the extract of Dendrobium officinale flower and/or the extract of Scrophularia ningpoensis and/or the extract of Eucommia ulmoides (each as described above), the botanical active component (and therefore the composition) further comprises, optionally further consists essentially of, or optionally further consists of at least one supplemental extract selected from the group consisting of: i) an extract of Phyllanthus emblica; ii) an extract of Sophora japonica; iii) an extract of Aronia; iv) an extract of Punica granatum; and v) combinations of i) to iv).

In certain embodiments, only one of the four supplemental extracts i) to iv) is present in the composition. In other embodiments, only two of the four supplemental extracts i) to iv) is present in the composition. In yet other embodiments, only three of the four supplemental extracts i) to iv) is present in the composition. In yet other embodiments still, all four of the supplemental extracts i) to iv) are present in the composition. Each of the individual supplemental extracts i) to iv) may be referred to simply as the “botanical extract” or collectively as the “botanical extracts” and are described in turn below.

Phyllanthus emblica

In some embodiments, the botanical active component, and thus the composition, comprises the extract of Phyllanthus emblica, i.e., an extract comprising, optionally consisting essentially of material from the fruit-bearing plant species Phyllanthus emblica. The Phyllanthus emblica extract is not particularly limited, and may comprise or be any extract or combination of extracts from a Phyllanthus emblica plant suitable for use in the embodiments herein.

Phyllanthus emblica has been reported to contain various bioactive components, such as antioxidants. Phyllanthus emblica may simply be referred to as P. emblica, or via various other names such as Phyllanthus emblica L., emblic myrobalan, myrobalan, Indian gooseberry, malacca tree, amla, Yu Gan Zi, or Fructus Phyllanthi.

Specific examples of Phyllanthus emblica extracts are known in art. As such, the Phyllanthus emblica extract may be purchased or otherwise obtained commercially from various sources, prepared (e.g. using any conventional extraction technique(s) known in the art, such as any of those described herein), or combinations thereof. In certain embodiments, the extract of Phyllanthus emblica is obtained by water extracting (or aqueous extracting) plant material of Phyllanthus emblica. In further or other embodiments, the extract of Phyllanthus emblica is obtained by alcohol extracting (e.g., ethanol extracting), plant material of Phyllanthus emblica.

As will be understood by those of skill in the art, Phyllanthus emblica is primarily cultivated for its fruit. As such, in various embodiments, the extract of Phyllanthus emblica is an extract of Phyllanthus emblica fruit or emblic leafflower fruit. Suitable extractions include those noted above, e.g., water and ethanol extractions of fruit. The fruit can be from one or more plants, and can be fresh, dried, or otherwise aged.

The Phyllanthus emblica extract may comprise material from any part of the plant, or combinations of parts, and is not limited to fruit extracts. For example, the Phyllanthus emblica extract may comprise material extracted from one or more parts of a Phyllanthus emblica plant, including the root, stem, bark, rhizome, leaf, bud, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Phyllanthus emblica extract. Likewise, the Phyllanthus emblica plant may be extracted in raw form, or processed prior to extraction of the Phyllanthus emblica extract (e.g. used in raw form, suspended form, dehydrated form, concentrated form, etc.). In certain embodiments, the botanical active component comprises a Phyllanthus emblica extract comprising material obtained (i.e., extracted) from fruit of Phyllanthus emblica.

By way of example, certain extracts can be obtained where Phyllanthus emblica (e.g. fruit) is pulverized to a homogeneous size in a mill. Next, the resulting powder is extracted using a water or ethanol solution. The solution is then filtered and the filtrate can be concentrated under reduced pressure to yield a syrup. The syrup can then be freeze-dried to dryness to obtain extract.

The amount of the Phyllanthus emblica extract utilized in the botanical active component may vary, and will be selected based on the number and types of components being utilized in the botanical active component. In certain embodiments, the botanical active component comprises from 1 to 2000 mg of the Phyllanthus emblica extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Phyllanthus emblica extract in an amount of at least 1, optionally of at least about 10, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Phyllanthus emblica extract in an amount of ≤100, ≤250, ≤500, ≤750, ≤1000, ≤2000, ≤5000 mg. In various embodiments, the botanical active component can include an amount of Phyllanthus emblica extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, ≤40, ≤50, ≤60, ≤70, ≤80, ≤90, and ≤99 wt. %, respectively, based on the total weight of the botanical active component.

In certain embodiments, the botanical active component comprises more than one Phyllanthus emblica extract, such as 2, 3, 4, or more Phyllanthus emblica extracts. In such embodiments, each Phyllanthus emblica extract is independently selected, may be the same as or different from any other Phyllanthus emblica extract, and each utilized in an amount as described above.

The Phyllanthus emblica extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Phyllanthus emblica extract(s) utilized. It will be appreciated that the Phyllanthus emblica extract may be combined with the carrier vehicle, if utilized, prior to, during, or after being combined with any other components of the botanical active component and/or composition.

Sophora japonica/Styphnolobium japonicum

In some embodiments, the botanical active component, and thus the composition, comprises the extract of Sophora japonica, i.e., an extract comprising, optionally consisting essentially of material from the flowering plant species Sophora japonica. The Sophora japonica extract is not particularly limited, and may comprise or be any extract or combination of extracts from a Sophora japonica plant suitable for use in the embodiments herein.

Sophora japonica has been reported to contain various bioactive components, such as the flavonoid glycosides sophoricoside, genistin and rutin, the flavonoid aglycones genistein, quercetin and kaempferol, genistein and the genistein glycosides including sophorabioside, sophoricoside, genistein-7-diglucoside, genistein-7-diglucorhamnoside, and kaempferol, and the kaempferol glycosides kaempferol-3-sophoroside and kaempferol-3-rhamnodiglucoside. Sophora japonica may simply be referred to as S. japonica, or via various other names such as Styphnolobium japonicum (L.) Schott, the pagoda tree, the Chinese scholar tree, and huai.

Specific examples of Sophora japonica extracts are known in art. As such, the Sophora japonica extract may be purchased or otherwise obtained commercially from various sources, prepared (e.g. using any conventional extraction technique(s) known in the art, such as any of those described herein), or combinations thereof. In certain embodiments, the extract of Sophora japonica is obtained by water extracting (or aqueous extracting) plant material of Sophora japonica. In further or other embodiments, the extract of Sophora japonica is obtained by alcohol extracting (e.g., ethanol extracting), plant material of Sophora japonica.

As will be understood by those of skill in the art, Sophora japonica is primarily cultivated for its flower. As such, in various embodiments, the extract of Sophora japonica is an extract of Sophora japonica flower. Suitable extractions include those noted above, e.g., water and ethanol extractions of flowers. The flowers can be from one or more plants, and can be fresh, dried, or otherwise aged.

The Sophora japonica extract may comprise material from any part of the plant, or combinations of parts, and is not limited to flower extracts. For example, the Sophora japonica extract may comprise material extracted from one or more parts of a Sophora japonica plant, including the root, stem, bark, rhizome, leaf, bud, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Sophora japonica extract. Likewise, the Sophora japonica plant may be extracted in raw form, or processed prior to extraction of the Sophora japonica extract (e.g. used in raw form, suspended form, dehydrated form, concentrated form, etc.). In certain embodiments, the botanical active component comprises a Sophora japonica extract comprising material obtained (i.e., extracted) from flowers of Sophora japonica.

By way of example, certain extracts can be obtained where Sophora japonica (e.g. flower) is pulverized to a homogeneous size in a mill. Next, the resulting powder is extracted using a water or ethanol solution. The solution is then filtered and the filtrate can be concentrated under reduced pressure to yield a syrup. The syrup can then be freeze-dried to dryness to obtain extract.

The amount of the Sophora japonica extract utilized in the botanical active component may vary, and will be selected based on the number and types of components being utilized in the botanical active component. In certain embodiments, the botanical active component comprises from 1 to 2000 mg of the Sophora japonica extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Sophora japonica extract in an amount of at least 1, optionally of at least about 10, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Sophora japonica extract in an amount of ≤100, ≤250, ≤500, ≤750, ≤1000, ≤2000, ≤5000 mg. In various embodiments, the botanical active component can include an amount of Sophora japonica extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, ≤40, ≤50, ≤60, ≤70, ≤80, ≤90, and ≤99 wt. %, respectively, based on the total weight of the botanical active component.

In certain embodiments, the botanical active component comprises more than one Sophora japonica extract, such as 2, 3, 4, or more Sophora japonica extracts. In such embodiments, each Sophora japonica extract is independently selected, may be the same as or different from any other Sophora japonica extract, and each utilized in an amount as described above.

The Sophora japonica extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Sophora japonica extract(s) utilized. It will be appreciated that the Sophora japonica extract may be combined with the carrier vehicle, if utilized, prior to, during, or after being combined with any other components of the botanical active component and/or composition.

### Aronia

In some embodiments, the botanical active component, and thus the composition, comprises the extract of Aronia, i.e., an extract comprising, optionally consisting essentially of material from the fruit-bearing plant genus Aronia. The Aronia extract is not particularly limited, and may comprise or be any extract or combination of extracts from a Aronia plant suitable for use in the embodiments herein.

Aronia has been reported to contain various bioactive components, including polyphenolic compounds, such as anthocyanins. Aronia is a genus of deciduous shrubs, the chokeberries. Chokeberries are different from chokecherries. The genus Aronia is considered to have three species: Aronia arbutifolia (or Photinia pyrifolia), which is also referred to simply as red chokeberry; Aronia melanocarpa (or Photinia melanocarpa), which is also referred to simply as black chokeberry; and Aronia prunifolia (or Photinia floribunda), which is also referred to simply as purple chokeberry. The black species generally contains higher levels of anthocyanins than the purple or red species, whereas the red and purple species are generally richer in phenolic acid and proanthocyanins.

Specific examples of Aronia extracts are known in art. As such, the Aronia extract may be purchased or otherwise obtained commercially from various sources, prepared (e.g. using any conventional extraction technique(s) known in the art, such as any of those described herein), or combinations thereof. In certain embodiments, the extract of Aronia is obtained by water extracting (or aqueous extracting) plant material of Aronia. In further or other embodiments, the extract of Aronia is obtained by alcohol extracting (e.g., ethanol extracting), plant material of Aronia.

As will be understood by those of skill in the art, Aronia is primarily cultivated for its fruit. As such, in various embodiments, the extract of Aronia is an extract of Aronia fruit. Suitable extractions include those noted above, e.g., water and ethanol extractions of fruit. The fruit can be from one or more plants, and can be fresh, dried, or otherwise aged.

The Aronia extract may comprise material from any part of the plant, or combinations of parts, and is not limited to fruit extracts. For example, the Aronia extract may comprise material extracted from one or more parts of a Aronia plant, including the root, stem, bark, rhizome, leaf, bud, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Aronia extract. Likewise, the Aronia plant may be extracted in raw form, or processed prior to extraction of the Aronia extract (e.g. used in raw form, suspended form, dehydrated form, concentrated form, etc.). In certain embodiments, the botanical active component comprises a Aronia extract comprising material obtained (i.e., extracted) from fruit of Aronia. In specific embodiments, the Aronia extract is simply Aronia fruit powder.

By way of example, certain extracts can be obtained where Aronia (e.g. fruit) is pulverized to a homogeneous size in a mill. Next, the resulting powder is extracted using a water or ethanol solution. The solution is then filtered and the filtrate can be concentrated under reduced pressure to yield a syrup. The syrup can then be freeze-dried to dryness to obtain extract.

The amount of the Aronia extract utilized in the botanical active component may vary, and will be selected based on the number and types of components being utilized in the botanical active component. In certain embodiments, the botanical active component comprises from 1 to 2000 mg of the Aronia extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Aronia extract in an amount of at least 1, optionally of at least about 10, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Aronia extract in an amount of ≤100, ≤250, ≤500, ≤750, ≤1000, ≤2000, ≤5000 mg. In various embodiments, the botanical active component can include an amount of Aronia extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, ≤40, ≤50, ≤60, ≤70, ≤80, ≤90, and ≤99 wt. %, respectively, based on the total weight of the botanical active component.

In certain embodiments, the botanical active component comprises more than one Aronia extract, such as 2, 3, 4, or more Aronia extracts. In such embodiments, each Aronia extract is independently selected, may be the same as or different from any other Aronia extract, and each utilized in an amount as described above.

The Aronia extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Aronia extract(s) utilized. It will be appreciated that the Aronia extract may be combined with the carrier vehicle, if utilized, prior to, during, or after being combined with any other components of the botanical active component and/or composition.

Punica granatum/Pomegranate

In some embodiments, the botanical active component, and thus the composition, comprises the extract of Punica granatum, i.e., an extract comprising, optionally consisting essentially of material from the fruit-bearing plant species Punica granatum. The Punica granatum extract is not particularly limited, and may comprise or be any extract or combination of extracts from a Punica granatum plant suitable for use in the embodiments herein.

Punica granatum has been reported to contain various bioactive components, including various phytochemicals. Punica granatum may simply be referred to as pomegranate or P. granatum, or via various other names such as Tian Shi Liu, Fructus Punicae Granati, or Shf Liu Pf. In general, the most abundant phytochemicals in pomegranate juice are polyphenols, including the hydrolyzable tannins called ellagitannins formed when ellagic acid and gallic acid bind with a carbohydrate to form pomegranate ellagitannins, also known as punicalagins. The red color of the juice is attributed to anthocyanins, such as delphinidin, cyanidin, and pelargonidin glycosides. Generally, an increase in juice pigmentation occurs during fruit ripening. Pomegranate peel contains high amount of polyphenols, condensed tannins, catechins, and prodelphinidins. Pomegranate seed oil contains punicic acid, palmitic acid, stearic acid, oleic acid, and linoleic acid.

Specific examples of Punica granatum extracts are known in art. As such, the Punica granatum extract may be purchased or otherwise obtained commercially from various sources, prepared (e.g. using any conventional extraction technique(s) known in the art, such as any of those described herein), or combinations thereof. In certain embodiments, the extract of Punica granatum is obtained by water extracting (or aqueous extracting) plant material of Punica granatum. In further or other embodiments, the extract of Punica granatum is obtained by alcohol extracting (e.g., ethanol extracting), plant material of Punica granatum.

As will be understood by those of skill in the art, Punica granatum is primarily cultivated for its fruit. As such, in various embodiments, the extract of Punica granatum is an extract of Punica granatum fruit. Suitable extractions include those noted above, e.g., water and ethanol extractions of fruit. The fruit can be from one or more plants, and can be fresh, dried, or otherwise aged.

The Punica granatum extract may comprise material from any part of the plant, or combinations of parts, and is not limited to fruit extracts. For example, the Punica granatum extract may comprise material extracted from one or more parts of a Punica granatum plant, including the root, stem, bark, rhizome, leaf, bud, flower, seed, and/or fruit, thereof. Moreover, such extracts may be further processed (e.g. defatted, partially defatted, ground, dried, precipitated, washed, filtered, mesh-sorted, extracted, distilled, concentrated, etc.) to obtain the Punica granatum extract. Likewise, the Punica granatum plant may be extracted in raw form, or processed prior to extraction of the Punica granatum extract (e.g. used in raw form, suspended form, dehydrated form, concentrated form, etc.). In certain embodiments, the botanical active component comprises a Punica granatum extract comprising material obtained (i.e., extracted) from fruit of Punica granatum. In specific embodiments, the Punica granatum extract is simply Punica granatum fruit powder.

By way of example, certain extracts can be obtained where Punica granatum (e.g. fruit) is pulverized to a homogeneous size in a mill. Next, the resulting powder is extracted using a water or ethanol solution. The solution is then filtered and the filtrate can be concentrated under reduced pressure to yield a syrup. The syrup can then be freeze-dried to dryness to obtain extract.

The amount of the Punica granatum extract utilized in the botanical active component may vary, and will be selected based on the number and types of components being utilized in the botanical active component. In certain embodiments, the botanical active component comprises from 1 to 2000 mg of the Punica granatum extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Punica granatum extract in an amount of at least 1, optionally of at least about 10, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Punica granatum extract in an amount of ≤100, ≤250, ≤500, ≤750, ≤1000, ≤2000, ≤5000 mg. In various embodiments, the botanical active component can include an amount of Punica granatum extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, 540, ≤50, ≤60, ≤70, ≤80, ≤90, and ≤99 wt. %, respectively, based on the total weight of the botanical active component.

In certain embodiments, the botanical active component comprises more than one Punica granatum extract, such as 2, 3, 4, or more Punica granatum extracts. In such embodiments, each Punica granatum extract is independently selected, may be the same as or different from any other Punica granatum extract, and each utilized in an amount as described above.

The Punica granatum extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Punica granatum extract(s) utilized. It will be appreciated that the Punica granatum extract may be combined with the carrier vehicle, if utilized, prior to, during, or after being combined with any other components of the botanical active component and/or composition.

### Definitions

In order to provide a clear and consistent understanding of the specification and claims, the following definitions are provided.

“Improving at least one sign of aging” and “improving a sign of aging” are used interchangeably herein to designate preventing, arresting, reversing, ameliorating, diminishing, and/or reducing a sign of aging. Representative signs of aging include, but are not limited to, lines, fine lines, wrinkles, crow's feet, dark eye circles, blemishes, age spots, stretch marks, or combinations thereof.

“Improving the appearance of skin” and “improving the aesthetic appearance of skin” are used interchangeably herein to designate an aesthetic improvement in the appearance of skin. Representative improvements may include, but are not limited to, favorable characteristics and/or properties related skin thickness, elasticity, resiliency, moisturization, smoothness, tone, texture, radiance, luster, brightness, clarity, contour, firmness, tautness, suppleness, softness, sensitivity, pore size, or combinations thereof. These terms may also be used to designate an improvement in an adverse skin condition. Representative adverse conditions affecting by, resulting in or resulting from such an adverse skin condition include, but are not limited to, psoriasis, eczema, seborrhea, dermatitis, sunburn, estrogen imbalance, hyperpigmentation, hypopigmentation, discoloration, yellowing, freckles, skin atrophy, skin breakout, skin fragility, dryness, tactile roughness, chapping, sagginess, thinning, hyperplasia, fibrosis, enlarged pores, cellulite formation, bruising, acne formation, apoptosis, cellular differentiation, cellular de-differentiation, prevention of tumor induction or tumor progression, viral infections, fungal infections, bacterial infections, spider veins (telangectasia), hirsutism, rosacea, pruritis, calluses, warts, corns, or combinations thereof.

The terms “composition” or “formulation” refer to a product that treats, improves, promotes, increases, manages, controls, maintains, optimizes, modifies, reduces, inhibits, or prevents a particular condition associated with a natural state, biological process or disease or disorder. For example, a composition or a formulation improves at least one sign of aging skin in a subject and/or minimizes or inhibits glycation in skin (e.g., mature skin). The terms composition and formulation include, but are not limited to, pharmaceutical (i.e., drug), over-the counter (OTC), cosmetic, food, food ingredient or dietary supplement compositions that include an effective amount of an extract, at least one component thereof, or a mixture thereof. Exemplary compositions and/or formulations include cream, cosmetic lotion, pack or powder, or as an emulsion, lotion, liniment foam, tablets, plasters, granules, or ointment. Preferred compositions are formulated for topical application/administration and for oral administration/ingestion.

As used herein, the term “effective amount” or “therapeutically effective amount” of a pure compound, composition, extract, extract mixture, component of the extract, and/or active agent or ingredient, or a combination thereof refers to an amount effective at dosages and for periods of time sufficient to achieve a desired result. For example, the “effective amount” or “therapeutically effective amount” refers to that amount of a pure compound, composition, extract, botanical extract, extract mixture, botanical extract mixture, component of the extract, and/or active agent or ingredient, or a combination thereof of this invention which, when administered to a subject (e.g., mammal, such as a human), is sufficient to effect treatment, such as improving aging skin and/or minimizing or inhibiting glycation in mature skin. The amount of a composition, extract, botanical extract, extract mixture, botanical extract mixture, component of the extract, and/or active agent or ingredient of this disclosure that constitutes an “effective amount” or “therapeutically effective treatment” will vary depending on the active agent or the compound, the condition being treated and its severity, the manner of administration, the duration of treatment, or the age of the subject to be treated, but can be determined routinely by one of ordinary skill in the art having regard to his own knowledge and to this disclosure.

The term “pharmaceutically acceptable” means those drugs, medicaments, extracts or inert ingredients, which are suitable for use in contact with the tissues of humans and lower animals without undue toxicity, incompatibility, instability, irritation, and the like, commensurate with a reasonable benefit/risk ratio.

The terms “applying” and “administering” are defined as providing a composition to a subject via a route known in the art, including but not limited to topical, intravenous, intra-arterial, oral, parenteral, buccal, transdermal, rectal, intramuscular, subcutaneous, intraosseous, transmucosal, or intraperitoneal routes of administration. In preferred embodiments, topical and/or oral routes of administering the described composition are suitable.

The terms “minimize,” “reduce,” “suppress,” “decrease” and/or “inhibit” refer to a decrease or reduction in protein activity and/or expression, and/or its downstream effect, in the presence of a botanical (or plant) ingredient or botanical extract as described herein, when compared to protein activity and/or expression in the absence of a botanical ingredient or botanical extract as described herein, such as in a control sample. The degree of decrease or inhibition of protein activity and/or expression, and/or its downstream effect, will vary with the nature and quantity of a botanical ingredient or botanical extract present, but will be evident, e.g., as a detectable decrease in protein activity and/or expression; desirably a degree of decrease greater than about 5%, about 10%, about 15%, about 20%, about 25%, about 50%, about 75%, about 90%, about 95% or about 99% (or any degree of decrease in the range of from about 5% to about 99%) as compared to protein activity and/or expression in the absence of the botanical ingredient or botanical extract. For example, a composition comprising a plant ingredient or plant extract of Dendrobium officinale can minimize or reduce glycation in skin, such as a mature skin.

As used herein, the term “subject” or “individual” includes mammals to which a composition may be administered. Non-limiting examples of mammals include humans, non-human primates, rodents (including transgenic and non-transgenic mice) or the like. In some embodiments, the subject is a mammal, and in some embodiments, the subject is human.

### Compositions (or Formulations)

The composition may include any amount of the botanical active component, which will be selected based on the number and types of components being utilized in the composition as a whole. In general, the botanical active component is present in the composition in an amount effective to inhibit AGEs in skin of the subject.

In certain embodiments, composition comprises the botanical active component in an amount of from 1 to 5000, optionally of from 2 to 2000 mg, optionally of from 5 to 1750, optionally of from 10 to 1500, optionally of from 15 to 1250, optionally of from 20 to 1000, optionally of from 25 to 750, optionally of from 30 to 500, optionally of from 35 to 500, optionally of from 40 to 500, optionally of from 45 to 450, optionally of from 50 to 450, or optionally of from 50 to 400, mg. However, amounts outside and/or overlapping with these ranges may also be utilized. For example, it is to be appreciated that the ranges described above with respect to the amount of each botanical extract in the botanical active component may equally apply to the amount of each botanical extract in the composition as a whole, such as when the botanical active component consists of but one of the botanical extracts.

In specific embodiments of the composition, the formulation comprises: flower of Dendrobium officinale Kimura et Migo extract; fruit of Phyllanthus emblica L. extract; flower of Sophora japonica L. extract; Aronia fruit powder; and pomegranate fruit powder. Further specific embodiments are illustrated in Table A below.

TABLE A
Exemplary Formulations

| | Formulation | | | |
|---|---|---|---|---|
| Component (mg) | No. 1 | No. 2 | No. 3 | No. 4 |
| Phyllanthus emblica | 250 | 300 | 200 | 200 |
| extract | | | | |
| Sophora japonica extract | 800 | 1000 | 600 | 600 |
| Aronia extract | 100 | 100 | 100 | 100 |
| Dendrobium officinale | 20 | 50 | 10 | 10 |
| flower extract | | | | |
| Pomegranate extract | 800 | 550 | 20 | 200 |
| Total plant extracts (mg) | 1970 | 2000 | 930 | 1110 |
| (Optional) Flavoring | 10-300 | 10-300 | 10-300 | 10-300 |
| essence | | | | |
| (Optional) Sugar | 5-3000 | 5-3000 | 5-3000 | 5-3000 |
| substitute | | | | |
| Total formulation weight | 1970-5270 | 2000-5300 | 930-4230 | 1110-4410 |
| (mg) | | | | |

In Table A above, the flavoring essence and sugar substitute can be any type of conventional component, e.g., flavoring agents, understood in the art. The plant (or botanical) extracts can each be as described above. Examples of suitable flavoring agents are described further below.

In general, the composition is not limited in terms of formulation, peripheral ingredients, form, number of functions, etc., aside from comprising the botanical active component and the botanical extract(s) thereof. Rather, the composition may be varied, and may be formulated in any fashion consistent with this disclosure.

Typically, the composition is formulated or otherwise adapted for administration to a mammalian subject (e.g. a human). For example, in various embodiments, the composition is adapted to be topically administrated or consumed and/or orally administered to a human subject.

In certain embodiments, the composition is further defined as a topical composition that is formulated for topical administration to the subject. In such embodiments, the composition may also be referred to as a cosmetic composition, and typically comprises at least one cosmetically acceptable carrier in addition to the bioactive agent composition. In specific embodiments, the cosmetically acceptable carrier is not naturally occurring. In other words, the carrier is not a product of nature in these specific embodiments. In other embodiments, the carrier is selected from conventional carriers understood in the art, and can be used in conventional amounts.

In other certain embodiments, the composition is further defined as an oral composition that is formulated for oral administration to the subject. In such embodiments, the composition may also be referred to as an ingestible composition, and typically comprises at least one pharmaceutically acceptable additive in addition to the bioactive agent composition. In specific embodiments, the pharmaceutically acceptable additive is not naturally occurring. In other words, the pharmaceutically acceptable additive is not a product of nature in these specific embodiments. In other embodiments, the pharmaceutically acceptable additive is selected from conventional additives understood in the art, and can be used in conventional amounts.

As such, it should be appreciated that the particular additives, carriers, adjuvants, fillers, etc. present in or combined with the composition may vary. Moreover, the physical form of the composition is not limited, and will be selected based on the particular components of the composition, a desired use of the composition, etc. As such, as will be understood in view of the description herein, the composition may be formulated as a liquid, dry powder, suspension, emulsion, gel, paste, etc., and combinations thereof. In certain embodiments, the composition is formulated as a sterile, non-pyrogenic liquid solution or suspension, a coated capsule, a suppository, a lyophilized powder, a transdermal patch, a softgel, or other forms are known. Other examples of suitable forms include solids, gels, liquids, creams, lotions, pomades, mousses, powders, foams, sprays, ointments, or other such preparations where the botanical active component is disposed in an appropriate carrier vehicle, such as any of those described herein. In particular embodiments, the composition is formulated or otherwise provided as an eye cream or mask for topical application.

The composition can be prepared using various methods. For example, actives of the composition (such as the botanical extract(s)), and optionally one or more inactives (such as one or more conventional components, additives, excipients, etc.), can be mixed or blended and compressed or compounded utilizing various techniques understood in the art. The composition of this disclosure is not limited to a particular order of manufacturing steps or method of manufacture.

In various embodiments, the composition is administered topically by application to the subject's skin. The subject is typically a human, and can include men and women of various ages. The method/composition of this disclosure is not limited to a particular subject.

The composition can be in various forms. Examples of suitable forms include solids, gels and liquids. For example, the composition can be formulated for application as a gel, cream, lotion, pomade, mousse, powder, or foam for application to the subject's skin. In another example, the composition can be formulated for spraying onto a subject's skin. The composition can be formulated to be sprayed as either an aerosol spray or pump spray. In still another example, the composition can be formulated for application using a pre-moistened towelette. In another example, the composition can be formulated as a solid that is rubbed onto the subject's skin. In another example, the composition is formulated for delivery through a patch that is adhered to the subject's skin.

Other than the botanical active component (i.e., the “actives” or “active ingredients”), the composition can include pharmaceutically acceptable additives that are inactives (or “inactive ingredients”) including, but not limited to, excipients, such as diluents and binders; granulating agents; glidants (or flow aids); fillers; lubricants; preservatives; stabilizers; coatings; disintegrants; fragrances; and pigments. The active ingredients and the pharmaceutically acceptable additives can be combined or compounded as desired to form an individual dose that provides the desired amount of active ingredient to the human subject when topically applied.

Optionally, the composition may include one or more additional components such as additives. Suitable additives include those understood in the art, including but not limited to, moisturizers, emollients, emulsifiers, surfactants, oils, extracts, skin protectants, disinfectants, antiseptics, drugs and drug substances, analgesic compounds, anti-neuralgic compound, antioxidants, blood circulation promoters, antidepressant compounds, anti-anxiety compounds, anti-stress compounds, sunscreens, insect repellants, preservatives, exfoliants, fragrances, colors, fillers, solvents, vehicles, carriers, other types of additives known to those of skill in the art, and combinations thereof. Such additives may be utilized alone or in combination. In general, the optional additives may be of any type used in personal care products and cosmetic products.

Excipients can be further classified as other components. Specifically, excipients used in oral solid dosage forms have been classified based on their functionality into groups such as diluents, disintegrants, binders, compression aids, granulating agents, glidants, lubricants, release-controlling polymers, stabilizers (such as antioxidants, chelators, and pH-modifiers), film-coating polymers, coating agents, vehicles, plasticizers, surfactants, colorants, sweeteners, and flavors.

In various embodiments, the composition comprises at least one component selected from the group consisting of binders, lubricants, glidants, and combinations thereof. In certain embodiments, the composition includes one or more compounds including, but not limited to, methylcellulose, hydroxypropyl methylcellulose, ethyl cellulose, cellulose acetate phthalate, acacia, gums, wax, glycerol monostearate, acrylic acid polymers and copolymers, methacrylic acid, methyl acrylate, ethyl acrylate, methyl methacrylate, ethyl methacrylate, lactose, calcium sulfate, calcium phosphate dibasic, sugar, microcrystalline cellulose (MCC), starch, sodium starch glycolate, polyvinylpyrrolidone, polyethylene glycol, and magnesium stearate. Combinations of such components can be utilized, and such components and other components used in conventional tablets are understood in the art.

As used herein, “diluents” may be inert substances added to increase the bulk of the composition to make a tablet a practical size for compression. As such, they may also be referred to as bulking agents. Commonly used diluents include, but are not limited to, microcrystalline cellulose (MCC), wood cellulose, corn starch, modified corn starch, (tri)calcium phosphate, calcium sulfate, lactose, kaolin, mannitol, sodium chloride, dry starch, (powdered) sugar, dextrose, mannitol, sorbitol, and the like. The diluent/bulking agent may be used alone or in various mixtures, and utilized in any amount known in the art for oral compositions.

As used herein, “flavoring agents” are compounds designed to give the composition a more palatable taste. Flavoring agents vary considerably in their chemical structure, ranging from simple esters, alcohols, and aldehydes to carbohydrates and complex volatile oils. Synthetic flavors of almost any desired type are now available and are well known in the art. If hard taste, acid taste or bitter taste derived from starting materials may be suppressed by seasoning or flavoring, the acidulant (e.g., citric acid, tartaric acid, malic acid, ascorbic acid, etc.), the sweetener (e.g. sodium saccharin, dipotassium glycyrrhizinate, aspartame, stevia, thaumatin, etc.), or the perfume (e.g. various fruit perfumes containing lemon oil, orange oil or strawberry, and yoghurt, mint, menthol, etc.) may be included in the composition. The flavoring agent may be used alone or in various mixtures, and utilized in any amount known in the art for oral compositions.

As used herein, “lubricants” are materials that perform a number of functions relating to compositions. In certain embodiments, like tablet manufacture, the lubricants perform one or more functions such as improving the rate of flow of the tablet granulation, preventing adhesion of the tablet material to the surface of dies and punches, reducing interparticle friction, and facilitating the ejection of the tablets from a die cavity. Examples of suitable lubricants include, but are not limited to, zinc stearate, gum arabic powder, cacao butter, carnauba wax, carmellosecalcium, carmellosesodium, caropeptide, aqueous silicon dioxide, dried aluminum hydroxide gel, glycerin, magnesium silicate, light anhydrous silicic acid, light liquid paraffin, crystalline cellulose, hardened oil, synthetic aluminum silicate, sesame oil, flour starch, white beeswax, magnesium oxide, dimethyl polysiloxane, potassium sodium tartrate, sucrose fatty acid ester, glycerin fatty acid ester, silicon resin, aluminum hydroxide gel, stearyl alcohol, stearic acid, aluminum stearate, calcium stearate, polyoxyl stearate, magnesium stearate, cetanol, gelatin, talc, magnesium carbonate, precipitated calcium carbonate, cornstarch, lactose, hard fat, saccharose, potato starch, hydroxypropylcellulose, fumaric acid, sodium stearyl fumarate, polyethylene glycol, polyoxyethylene polyoxypropylene glycol, polysorbate, beeswax, magnesium aluminometasilicate, methylcellulose, Japan wax, glycerin monostearate, sodium lauryl sulfate, calcium sulfate, magnesium sulfate, liquid paraffin, phosphoric acid, palmitic acid, and hydrogenated vegetable oils and fats. The lubricant may be used alone or in various mixtures, and utilized in any amount known in the art for oral compositions.

As used herein, “binders” are agents used to impart cohesive qualities to powdered materials. Binders, or “granulators” as they are sometimes known, impart a cohesiveness to the tablet formulation, which ensures the tablet remaining intact after compression, as well as improving the free-flowing qualities by the formulation of granules of desired hardness and size. Materials commonly used as binders include starch, such as corn starch and pregelatinized starch; gelatin; sugars, such as sucrose, glucose, dextrose, molasses, and lactose; natural and synthetic gums, such as gum acacia, sodium alginate, extract of Irish moss, panwar gum, ghatti gum, mucilage of isapol husks, carboxymethylcellulose, methylcellulose, polyvinylpyrrolidone (PVP), Veegum, microcrystalline cellulose, microcrystalline dextrose, amylose, larch arabogalactan, ethyl cellulose, cellulose acetate, and the like. The binder may be used alone or in various mixtures, and utilized in any amount known in the art for oral compositions.

As used herein, “colorants” are agents that give the composition a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye. The colorant may be used alone or in various mixtures, and utilized in any amount known in the art for oral compositions.

Other conventional ingredients that may optionally be present in the composition include preservatives, stabilizers, anti-adherents or silica flow conditioners or glidants, such as silicon dioxide. Such ingredients may be used alone or in various mixtures, and utilized in any amount known in the art for oral compositions.

It is to be appreciated that certain components or additives may be classified under different terms of art and just because a component or additive is classified under such a term does not mean that they are limited to that function. If utilized, the additive or additives may be present in the composition in various amounts. Additional ingredients for optional use in the composition, e.g. when adapted for topical or oral administration, are described in U.S. Pat. Nos. 5,747,006; 5,980,904; 6,994,874; 7,060,304; 7,247,321; 7,348,034; 7,364,759; 7,700,110; 7,722,904; 8,202,556; 8,916,212; 9,445,975; 9,801,809; 10,307,366; 10,532,024; and 10,537,516; and in U.S. Publication Nos. 2006/0257509; 2007/0224154; 2008/0081082; 2008/0124409; 2013/0302265; 2017/0252293; 2017/0281666; 2018/0200285; 2019/0083566; 2019/0160117; 2020/0171117; 2020/0383898; 2021/0017240; and 2021/0212926; the disclosures of which are hereby incorporated by reference in their entirety.

### Method of Administration

The composition may be administered or applied as needed, daily, several times per day or in any suitable regimen such that the desired outcome is achieved. In the method of this disclosure, the frequency of administration (e.g. topical application) can depend on several factors, including the desired level of AGE inhibition. Generally, a regimen includes application of the composition once or twice daily to include an administration in the morning and/or an administration in the evening. The amount and/or frequency of application of the composition may depend on several factors, including the level of desired results and the specific composition.

Improved skin appearance can be achieved by administering the formulations of the present invention externally, internally, or some combination thereof. Preferably, the formulations of the present invention are administered with an acceptable carrier. For example, the formulation of the present invention could be externally administered with an acceptable carrier in the form of a gel, lotion, cream, tonic, emulsion, etc. As a further example, the formulation of the present invention could be internally administered with an acceptable carrier in the form of a pill, tablet, powder, bar, beverage, etc. Thus, the formulations described herein are useful in a wide variety of finished products, including pharmaceutical products, food products, and beverage compositions. Preferably, the products are useful for providing mammalian skin with an improved appearance.

When the formulations of the present invention are orally administered in the form of a liquid, the liquid may be water-based, milk-based, tea-based, fruit juice-based, or some combination thereof. Solid and liquid formulations for internal administration according to the present invention can further comprise thickeners, including xanthan gum, carboxymethylcellulose, carboxyethylcellulose, hydroxypropylcellulose, methylcellulose, microcrystalline cellulose, starches, dextrins, fermented whey, tofu, maltodextrins, polyols, including sugar alcohols (e.g., sorbitol and mannitol), carbohydrates (e.g. lactose), propylene glycol alginate, gellan gum, guar, pectin, tragacanth gum, gum acacia, locust bean gum, gum arabic, gelatin, as well as mixtures of these thickeners. These thickeners are typically included in the formulations of the present invention at levels up to about 0.1%, depending on the particular thickener involved and the viscosity effects desired.

The solid and liquid (food and beverage) formulations of the present invention can, and typically will, contain an effective amount of one or more sweeteners, including carbohydrate sweeteners and natural and/or artificial no/low calorie sweeteners. The amount of the sweetener used in the formulations of the present invention will vary, but typically depends on the type of sweetener used and the sweetness intensity desired.

In another example, the formulations of the present invention are topically administered in the form of a: solution, gel, lotion, cream, ointment, oil-in-water emulsion, water-in-oil emulsion, stick, spray, paste, mousse, tonic, foundation, or other cosmetically and topically suitable form.

Preferably, formulations of the present invention that are suitable for topical administration are mixed with an acceptable carrier. An acceptable carrier may act variously as solvent, carrier, diluent or dispersant for the constituents of the composition, and allows for the uniform application of the constituents to the surface of the skin at an appropriate dilution. The acceptable carrier may also facilitate penetration of the composition into the skin.

In one example of a formulation for topical application, the acceptable carrier forms from about 70% to about 99.99% by weight of the total composition. In other examples, the acceptable carrier will form from about 85% to 99.99% by weight of the total composition. The acceptable carrier may also form from about 90% to about 99.99% by weight of the total composition; or from about 99.95% to about 99.999% by weight of the total composition. The acceptable carrier can, in the absence of other cosmetic adjuncts or additives, form the balance of the composition.

The various ingredients used in practicing the present invention may be soluble or insoluble in the acceptable carrier. If all ingredients of a formulation are soluble in the acceptable carrier, then the vehicle acts as solvent. However, if all or some ingredients of a formulation are insoluble in the acceptable carrier, then those ingredients are dispersed in the vehicle by means of, for example, a suspension, emulsion, gel, cream or paste, and the like.

Thus, it will be apparent to the skilled artisan that the range of possible acceptable carriers is very broad. For example, acceptable carriers can be emulsions, lotions, creams, or tonics. Acceptable carriers can comprise water, ethanol, butylene glycol, or other various solvents that aid in penetration of the skin. Some examples of suitable vehicles are described in U.S. Pat. Nos. 6,184,247 and 6,579,516, the entire contents of which are incorporated herein by reference.

Preferably the acceptable carrier used in practicing the present invention comprises water and ethanol. Optionally, the acceptable carrier also contains butylene glycol. For example, the acceptable carrier can comprise 2-5% butylene glycol by weight of the composition. In practicing the present invention, preferably this acceptable carrier is mixed with a formulation of the present invention comprising 2% by weight of the total composition. In other examples, the acceptable carrier is mixed with a formulation of the present invention comprising 0.001% to 30% by weight of the total composition; 1% to 5% by weight of the total composition; 0.01% to 15% by weight of the total composition; or 0.5% to 1.0% by weight of the total composition.

In general, however, acceptable carriers according to the present invention may comprise, but are not limited to comprising, any of the following examples: water; castor oil; ethylene glycol monobutyl ether; diethylene glycol monoethyl ether; corn oil; dimethyl sulfoxide; ethylene glycol; isopropanol; soybean oil; glycerin; soluble collagen; safflower seed oil; meadowfoam seed oil; mineral oil; squalene; shea butter; borage oil; or rice bran oil; polyquaternium-10; methylparaben; PEG-8; disodium lauroamphodacetate; sodium trideceth sulfate; hexylene glycol; sodium methyl cocoyl taurate; tea-lauryl sulfate; lauryl betaine; sodium myristoyl sarcosinate; PEG-150 distearate; citric acid-anhydrous; sodium citrate-dihydrate; diazolidinyl urea; disodium EDTA; propylparaben; polysorbate 60; isopropyl palmitate; octyl palmitate; C12-15 alkyl benzoate; dipropylene glycol dibenzoate; PPG-15 stearyl ether benzoate; isododecane; isoeicosane; squalane; jojoba oil; dimethicone; glyceryl stearate; PEG-100 stearate; cetyl alcohol; butylene glycol; chlorphenesin; fragrance; polyacrylamide; C13-14 isoparaffin; Laureth-7; aloe vera powder; aloe vera gel, hydroxyethylacrylate; sodium acryloyldimethyl taurate copolymer; behenyl alcohol; tocopheryl acetate; isodecyl neopentanoate; glyceryl trioctanoate; cetearyl alcohol; cetearyl glucoside; Chamomilla recutita flower extract; biosaccharide gum-1; pentadecalactone; dipropylene glycol; cyclomethicone; PEG/PPG-18/18 Dimethicone; cyclopentasiloxane; disteardimonium hectorite; SD alcohol 40; phenoxyethanol; ethylparaben; trimethylsiloxysilicate; triethoxycaprylysilane; micronized titanium dioxide; titanium dioxide; zinc oxide; iron oxides (yellow; red; black; etc.); caprylysilane; sodium chloride; diisopropyl dimer dilinoleate; aluminum hydroxide; stearic acid; polyethelene beads; C12-15 alkyl benzoate; acrylates/C10-30 alkyl acrylate; xanthan gum; sorbitan laurate; panthenol; petrolatum; isopropyl isostearate; dimethicone; arginine; phenoxyethanol; acryloyldimethyl taurate copolymer; isohexadecane; polysorbate 80; hydroxyethylacrylate; sodium acryloyldimethyl taurate copolymer; octinoxate (octyl methoxycinnimate); oxybenzone; dicaprylyl ether; isodecyl neopentanoate; cetearyl alcohol; cetearyl glucoside; benzyl alcohol; HDI/trimethylol hexyllactone crosspolymer; silica; isodecyl neopentanoate; coco-glucoside; C20-22 alkyl phosphate; C20-22 alcohols; palmitoyl proline; magnesium palmitoyl glutamate; sodium palmitoyl sarcosinate; C30-45 alkyl cetearyl crosspolymer; polyacrylate 13; polyisobutene; polysorbate 20; iodopropynyl butylcarbamate; sodium magnesium silicate; methyl gluceth-20; dimethyl isosorbide; silica; SD alcohol 40-B; salicylic acid; ceteth-20; fragrance; or witch hazel.

Additionally, acceptable carriers used in the present invention may optionally comprise one or more humectants, including but not limited to: dibutyl phthalate; soluble collagen; sorbitol; or sodium 2-pyrrolidone-5-carboxylate. Other examples of humectants that may be used in practicing the present invention can be found in the CFTA Cosmetic Ingredient Handbook, the relevant portions of which are incorporated herein by reference.

Additionally, acceptable carriers in the present invention may optionally comprise one or more emollients including but not limited to: butane-1,3-diol; cetyl palmitate; dimethylpolysiloxane; glyceryl monoricinoleate; glyceryl monostearate; isobutyl palmitate; isocetyl stearate; isopropyl palmitate; isopropyl stearate; butyl stearate; isopropyl laurate; hexyl laurate; decyl oleate; isopropyl myristate; lauryl lactate; octadecan-2-ol; caprylic triglyceride; capric triglyceride; polyethylene glycol; propane-1,2-diol; triethylene glycol; sesame oil; coconut oil; safflower oil; isoamyl laurate; nonoxynol-9; panthenol; hydrogenated vegetable oil; tocopheryl acetate; tocopheryl linoleate; allantoin; propylene glycol; arachis oil; castor oil; isostearic acid; palmitic acid; isopropyl linoleate; lauryl lactate; myristyl lactate; decyl oleate; or myristyl myristate. Other examples of emollients that may be used in practicing the present invention can be found in the CFTA Cosmetic Ingredient Handbook, the relevant portions of which are incorporated herein by reference.

Additionally, acceptable carriers used in the present invention may optionally comprise one or more penetration enhancers including but not limited to: pyrrolidones, for example 2-pyrrolidone; alcohols, such as ethanol; alkanols, such as decanol; glycols, such as propylene glycol, dipropylene glycol, butylene glycol; surfactants; or terpenes.

Other acceptable carriers that may be used in practicing the present invention will be apparent to those of skill in the art and are included within the scope of the present invention.

For example, an acceptable carrier can be a lotion that is topically applied. The lotion may comprise cabomer 981, water, glycerin, isopropyl myristate, mineral oil, shea butter, stearic acid, glycol stearate, cetyl alcohol, dimethicone, preservatives, tea, and various ingredients of the formulations of the present invention.

The formulations of the present invention may also contain various known and conventional cosmetic adjuvants so long as they do not detrimentally affect the desired skin improvement and moisturizing effects provided by the formulation. For example, a formulation of the present invention can further include one or more additives or other optional ingredients well known in the art, which can include but are not limited to fillers (e.g., solid, semi-solid, liquid, etc.); carriers; diluents; thickening agents; gelling agents; vitamins, retinoids, and retinols (e.g., vitamin B₃, vitamin A, etc.); pigments; fragrances; sunscreens and sunblocks; antioxidants and radical scavengers; organic hydroxy acids; exfoliants; skin conditioners; moisturizers; ceramides, pseudoceramides, phospholipids, sphingolipids, cholesterol, glucosamine, pharmaceutically acceptable penetrating agents (e.g., n-decylmethyl sulfoxide, lecithin organogels, tyrosine, lysine, etc.); preservatives; antimicrobial agents; amino acids such as proline, pyrrolidone carboxylic acid, its derivatives and salts, saccharide isomerate, panthenol, buffers together with a base such as triethanolamine or sodium hydroxide; waxes, such as beeswax, ozokerite wax, paraffin wax; plant extracts, such as Aloe Vera, cornflower, witch hazel, elderflower, or cucumber and combinations thereof. Other suitable additives and/or adjuncts are described in U.S. Pat. No. 6,184,247, the entire contents of which are incorporated herein by reference.

The formulation can include additional inactive ingredients, including, but not limited to surfactants, co-solvents, and excipients. Surfactants, such as hydrophilic and hydrophobic surfactants, can be included in the formulations. Particular surfactants can be used based on the on the overall composition of the formulation and the intended delivery of the formulation. Useful surfactants include polyethoxylated (PEG) fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, polysaccharide esters, polyethylene glycol alkyl phenols, polyoxyethylene-polyoxypropylene block copolymers, sorbitan fatty acid esters, lower alcohol fatty acid esters, ionic surfactants, and mixtures thereof.

The formulations can also include co-solvents such as alcohols and polyols, polyethylene glycols ethers, amides, esters, other suitable co-solvents, and mixtures thereof. The formulations can also include excipients or additives such as sweeteners, flavorants, colorants, antioxidants, preservatives, chelating agents, viscomodulators, tonicifiers, odorants, opacifiers, suspending agents, binders, and mixtures thereof.

Generally, the formulations of the present invention are topically or orally administered at least on a daily basis for a period of time sufficient to bring about the desired level of improvement in skin appearance. Topical application or oral administration of the formulations of the invention may continue for any suitable period of time. More specifically, within a few hours to within a few days of the initial application or ingestion, a user may notice the skin has an improved appearance. It should be appreciated that the frequency with which the formulations of the present invention should be applied or ingested will vary depending on the desired level improved appearance. In particular, the degree of cosmetic enhancement will vary directly with the total amount of composition used.

Useful dosage forms can be prepared by methods and techniques that will be well understood by those of skill in the art and may include the use of additional ingredients in producing tablets, capsules, or liquid dosage forms.

### INDUSTRIAL APPLICABILITY

This disclosure provides new and useful advanced glycation end-product (AGE) inhibitors, including the botanical extracts described herein. Such AGE inhibitors can offer potential preventive and therapeutic approaches for lowering the risks of diabetic or other pathogenic complications caused by AGE formation. As such, the compositions and methods described herein are useful for inhibiting AGEs.

General compositions and product lines provided by this disclosure relate to skin care and nutrition-beauty products utilizing such AGE inhibitors, and specific examples include eye creams and masks utilizing such AGE inhibitors.

Left unchecked, glycation can result in significant accumulation of AGEs in long-lived macromolecules, such as collagen, where the amino acids lysine and arginine can undergo glycation and form cross-links, reducing skin elasticity, increasing the appearance of wrinkles, and causing healing deficiencies. Thus, the compositions and methods herein are useful for glycation inhibition or skin aging prevention, and therefore can increase skin elasticity, decrease the appearance of wrinkles, and otherwise promote skin healing. This compositions and methods of this disclosure also provide potential preventive and therapeutic approaches for lowering the risks of diabetic or other pathogenic complications caused by AGE formation.

The following examples, illustrating the compositions and methods of this disclosure, are intended to illustrate and not to limit the disclosure.

### EXAMPLES

### Initial Studies

The inhibition effects of five botanic water and ethanol extracts on protein non-enzymatic glycation was evaluated. Specifically, two extracts of Dendrobium officinale flower, one extract of Dendrobium officinale stem, one extract of Scrophularia ningpoensis Hemsl. radix, and one extract of Eucommia ulmoides male flowers were evaluated for their potential to inhibit AGEs formation. Four of the five extracts showed inhibition as illustrated in the following Tables and Figures. The extract of Dendrobium officinale stem did not show inhibition. The exacts were made in lab, but are also commercially available.

### Non-Enzymatic Glycation of HSA

The anti-glycative ability of samples were assessed by measuring the amounts of fluorescent AGEs. 100 μL human serum albumin (4 mg/ml HSA) was glycated with 100 μL D-fructose (0.05 mol/L) and 100 μL glucose (0.05 mol/L) in the presence of samples in potassium phosphate buffer (0.2 mol/L, pH7.4, 0.06% NaN₃) and incubated at 50° C. for 24. The incubated solutions were then used for the following assays. Rutin was the positive control.

### Determination of Fluorescent AGEs

The fluorescence intensity of the incubated solutions under the λex/λem=360/453 nm was recorded on microplate reader. The inhibition rate of fluorescent AGEs was calculated by the following equation:

$$The inhibition rate ( \% ) = \frac{( A 1 - A 2 ) - ( A 3 - A 4 )}{( A 1 - A 2 )} \times 100$$

where A1 is the fluorescence intensity of glycation system without samples; A2 is the fluorescence intensity of glycation system without HSA and samples; A3 is the fluorescence intensity of glycation system with HSA, sugar and samples; and A4 is the fluorescence intensity of glycation system without HSA.

The results are illustrated in the Tables below. The average rates of inhibition can also be appreciated with reference to the Figures.

TABLE 1
Rutin

| | Concentration (μg/ml) | | | | | | | |
|---|---|---|---|---|---|---|---|---|
| | 400 | 200 | 100 | 50 | 25 | 12.5 | 6.25 | 3.125 |
| The inhibition | 90.91 | 84.54 | 81.88 | 74.72 | 55.85 | 35.84 | 18.13 | 2.34 |
| rate of | 91.13 | 85.94 | 81.51 | 79.59 | 64.92 | 42.05 | 25.69 | −3.34 |
| fluorescent AGE | 91.53 | 86.58 | 82.32 | 73.08 | 64.97 | 45.57 | 22.17 | 7.07 |
| 360/453 nm (%) | | | | | | | | |
| Average % | 91.19 | 85.69 | 81.90 | 75.80 | 61.91 | 41.15 | 22.00 | 2.02 |
| inhibition* | | | | | | | | |

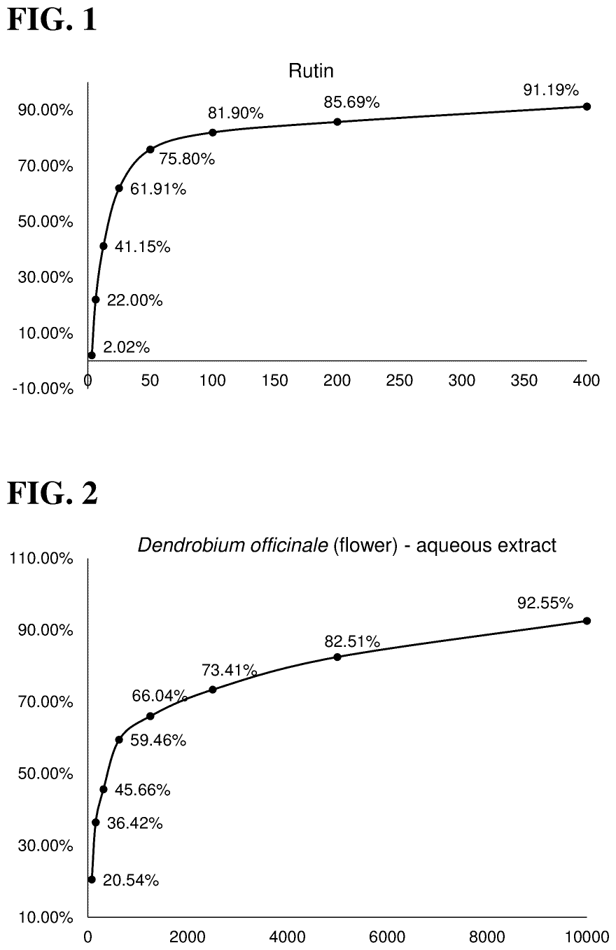

*see also FIG. 1

TABLE 2
Dendrobium officinale (flower) - aqueous extract

| | Concentration (μg/mL) | | | | | | | |
|---|---|---|---|---|---|---|---|---|
| | 10000 | 5000 | 2500 | 1250 | 625 | 312.5 | 156.25 | 78.125 |
| The inhibition | 94.19 | 79.16 | 69.56 | 65.94 | 53.90 | 43.17 | 34.49 | 17.32 |
| rate of | 90.92 | 90.95 | 78.38 | 66.83 | 67.17 | 44.66 | 35.67 | 21.51 |
| fluorescent AGE | 92.54 | 77.42 | 72.30 | 65.34 | 57.32 | 49.14 | 39.08 | 22.77 |
| 360/453 nm (%) | | | | | | | | |
| Average % | 92.55 | 82.51 | 73.41 | 66.04 | 59.46 | 45.66 | 36.41 | 20.53 |
| inhibition* | | | | | | | | |

*see also FIG. 2

TABLE 3
Dendrobium officinale (flower) - ethanol extract

| | Concentration (μg/mL) | | | | | | | |
|---|---|---|---|---|---|---|---|---|
| | 10000 | 5000 | 2500 | 1250 | 625 | 312.5 | 156.25 | 78.125 |
| The inhibition | 80.17 | 63.84 | 60.23 | 56.81 | 47.15 | 33.89 | 24.63 | −9.0 |
| rate of | 79.71 | 84.78 | 72.65 | 63.83 | 57.49 | 27.93 | −48.12 | −70.09 |
| fluorescent AGE | 81.57 | 70.43 | 75.18 | 65.43 | 50.38 | 29.67 | 11.36 | −84.58 |
| 360/453 nm (%) | | | | | | | | |
| Average % | 80.48 | 73.02 | 69.35 | 62.02 | 51.67 | 30.50 | −4.04 | −54.56 |
| inhibition* | | | | | | | | |

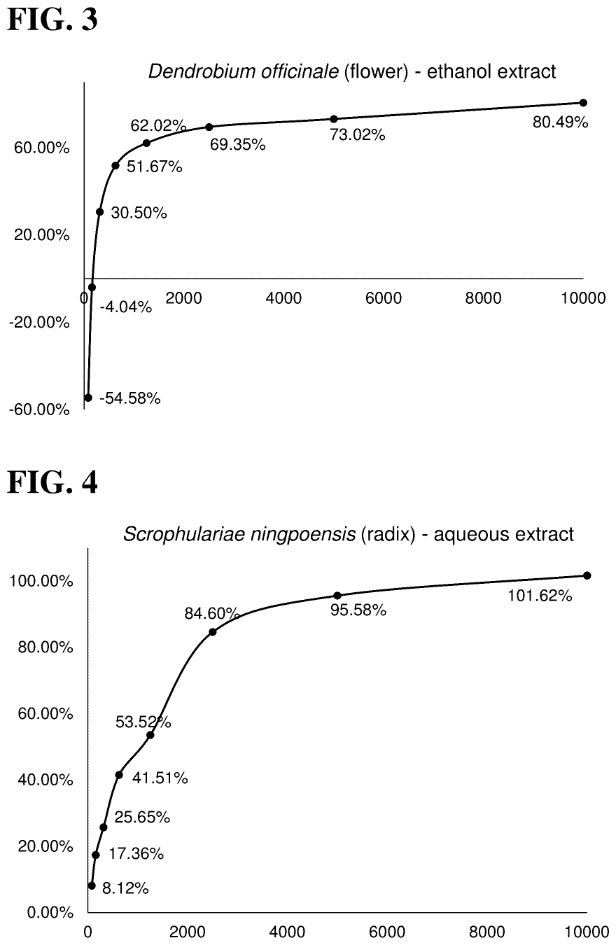

*see also FIG. 3

TABLE 4
Scrophularia ningpoensis Hemsl. (radix) - aqueous extract

| | Concentration (μg/ml) | | | | | | | |
|---|---|---|---|---|---|---|---|---|
| | 10000 | 5000 | 2500 | 1250 | 625 | 312.5 | 156.25 | 78.125 |
| The inhibition rate | 103.55 | 81.62 | 88.37 | 48.83 | 33.54 | 22.44 | 15.39 | 8.79 |
| of fluorescent AGE | 101.63 | 114.41 | 82.08 | 57.48 | 47.87 | 26.60 | 19.24 | 4.55 |
| 360/453 nm (%) | 99.68 | 90.72 | 83.35 | 54.25 | 43.12 | 27.91 | 17.44 | 11.03 |
| Average % | 101.62 | 95.58 | 84.60 | 53.52 | 41.51 | 25.65 | 17.36 | 8.12 |
| inhibition* | | | | | | | | |

*see also FIG. 4

TABLE 5
Eucommia ulmoides (male flower) - aqueous extract

| | Concentration (μg/ml) | | | | | | | |
|---|---|---|---|---|---|---|---|---|
| | 10000 | 5000 | 2500 | 1250 | 625 | 312.5 | 156.25 | 78.125 |
| The inhibition rate | 94.00 | 80.59 | 69.94 | 53.83 | 52.75 | 59.91 | 55.59 | 45.91 |
| of fluorescent AGE | 100.95 | 100.29 | 70.74 | 55.71 | 63.52 | 56.27 | 32.54 | 38.23 |
| 360/453 nm (%) | 99.40 | 85.02 | 74.46 | 50.60 | 46.89 | 57.46 | 65.02 | 44.47 |
| Average % | 98.12 | 88.63 | 71.71 | 53.38 | 54.39 | 57.88 | 51.05 | 42.87 |
| inhibition* | | | | | | | | |

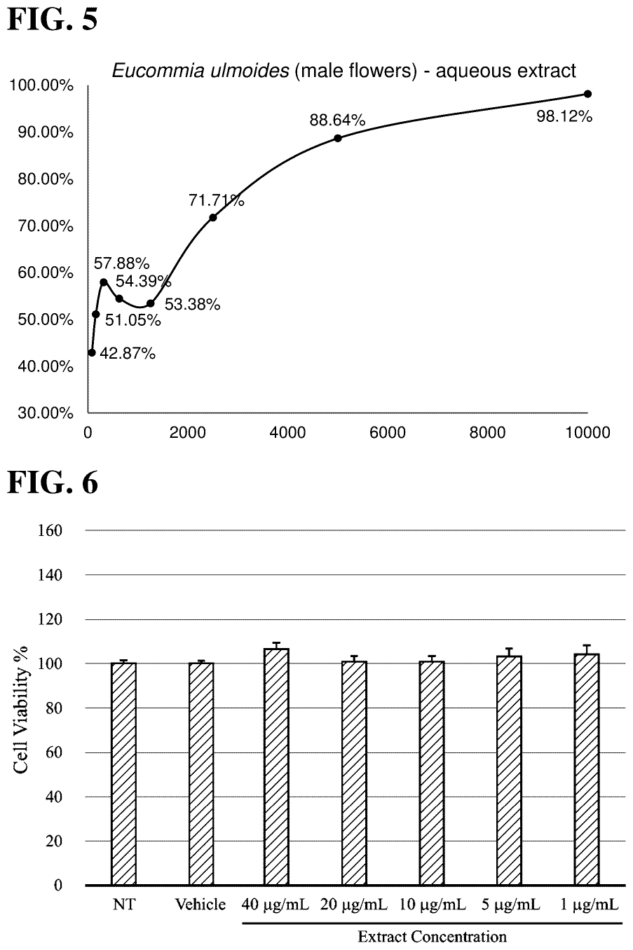

*see also FIG. 5

TABLE 6
Dendrobium officinale (stem) - aqueous extract

| | Concentration (μg/mL) | | | | | |
|---|---|---|---|---|---|---|
| | 2000 | 1000 | 500 | 250 | 125 | 62.5 |
| The inhibition rate of | 26.70 | 26.08 | 35.08 | 22.59 | 10.03 | 1.97 |
| fluorescent AGE | 38.71 | 33.26 | 37.88 | 33.18 | 20.26 | 4.25 |
| 360/453 nm (%) | 21.90 | 38.57 | 33.92 | 27.77 | 14.76 | 5.12 |
| Average % inhibition | 29.10 | 32.64 | 35.62 | 27.85 | 15.02 | 3.78 |

As shown in Table 6 above, stem extracts of Dendrobium officinale had little effect on anti-glycation activity. Thus, it was surprisingly discovered that Dendrobium officinale flowers are more effective relative to Dendrobium officinale stems to provide anti-glycation. It is believed that that difference in effect is based on polysaccharide content in the stems, which is mainly extracted by water.

### Evaluation of Anti-Glycation Activity on Primary Human Dermal Fibroblasts (HDFs) Cells

Dendrobium officinale (flower)-aqueous extract was stored at 4° C. before use. Primary HDFs were maintained in Dulbecco's Modified Eagle Medium (DMEM) containing 10% heat-inactivated Fetal Bovine Serum (FBS) and 1% penicillin/streptomycin at 37° C. under a humidified atmosphere of 95% air and 5% CO₂.

HDFs cells were digested from T75 flasks and seeded to several 96 well plates at the density of 10,000 cells per well. When reached 90% confluence in the wells and cells were treated with different concentrations of active sample (n=3). After 48 h the cells were washed once in Dulbecco's phosphate-buffered saline (DPBS) and the viability was then tested by CCK-8 kit according to the manufactural guidance. HDFs were further incubated for 2 h at 37° C. Final readings were performed by measuring the absorbance at 450 nm using a microplate reader.

### Evaluation of Anti-Glycation Activity

HDFs cells were digested from T75 flasks and seeded to 12 well culture plate on slides at 50,000 cells per well. After 48 h the cells were treated by 0.5 mM glyoxal for 48 h. Then the cells were treated with or without positive controls or active samples for an additional 48 h. At the end of the experiment, cell slides were washed once by phosphate-buffered saline (PBS) and fixed in polyformaldehyde overnight at 4° C. For chronic myeloid leukemia (CML) biomarker staining, the slides were blocked in 1% bovine serum albumin (BSA) for 1 h then applied with primary CML antibody (1:50) overnight at 4° C. The next day secondary antibody (1:1000) was applied for 2 h at room temperature. The slides were mounted by DAPI (4′, 6-diamidino-2-phenylindole) and relevant fluorescent images were captured by microscope. Image J was used to quantified CML expression from different visions randomly picked by each group. Data is presented as the mean±SD, and analyzed with Student's t-test or one-way ANOVA. * P<0.05, ** P<0.01, *** P<0.001.

TABLE 7
Effect of Samples on HDFs Cell Viability

| | | |
|---|---|---|
| Treatment | AVE | SD |

| No Treatment (NT) | | | 100.00 | 1.56 |
|---|---|---|---|---|
| Vehicle | | | 100.48 | 0.71 |
| | Dendrobium officinale | 40 μg/mL | 106.68 | 2.69 |
| | (flower) - aqueous extract* | 20 μg/mL | 100.57 | 2.56 |
| | (concentration) | 10 μg/mL | 100.76 | 2.71 |
| | | 5 μg/mL | 103.24 | 3.49 |
| | | 1 μg/mL | 104.20 | 3.98 |

*see also FIG. 6

As shown in Table 7 above, there was no cytotoxicity found in cells treated with different concentrations of Dendrobium officinale (flower)-aqueous extract. “AVE” is average and “SD” is standard deviation.

TABLE 8
Anti-glycation Effect of Dendrobium officinale
(flower) - aqueous extract

| * | Treatment | AVE | SD |
|---|---|---|---|
| (A) | No Treatment (NT) | 17.25 | 8.35 |
| (B) | Glyoxal | 100.00 | 13.39 |
| (C) | Aminoguanidine Hydrochloride (AG) - 0.1 mM | 11.89 | 5.62 |
| (D) | Dendrobium officinale (flower) - | 30.30 | 12.40 |
| | aqueous extract (40 μg/mL) | | |
| (E) | Dendrobium officinale (flower) - | 22.12 | 6.47 |
| | aqueous extract (10 μg/mL) | | |

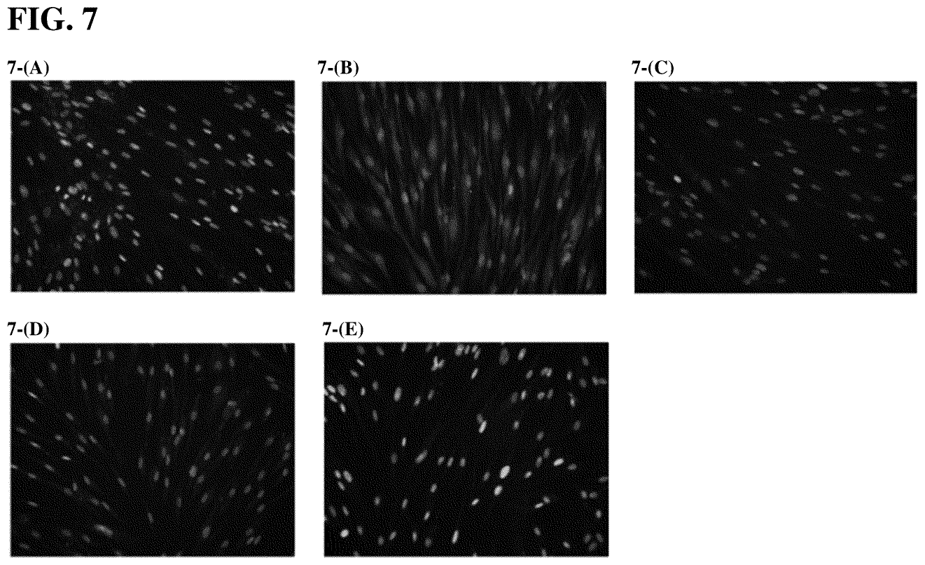

* see also FIG. 7

As shown in Table 8 above, Dendrobium officinale (flower)-aqueous extract provides excellent anti-glycation effect on HDFs.

### Evaluation of Anti-Glycation Activity on Zebrafish

Zebrafish were all fed in fish culture water at 28° C. (water quality: 200 mg instant sea salt was added to 1 L reverse osmosis water, the conductivity was 450-550 μS/cm; PH 6.5~8.5; Hardness of 50-100 mg/L CaCO₃), provided by a fish breeding center, experimental animal use license number: SYXK (Zhejiang) 2012-0171, feeding management in line with the international AAALAC certification (certification number: 001458) requirements. Wild type AB zebrafish are bred naturally in pairs. Zebrafish aged 5 days after fertilization (DPF) were used to evaluate the anti-aging efficacy of samples.

Five DPF wild type AB zebrafish were randomly selected and placed in 1.5 mL centrifuge tubes, with 10 zebrafish in each tube (experimental group). The extracts of Dendrobium officinale flower were given in aqueous solution with positive control aminoguanidine hydrochloride (AG) concentration of 200 μg/mL, and normal control group and model control group were set at the same time. The volume of each tube was 150 μL. Except for normal control group, the other experimental groups were given 0.4 M glucose solution to establish AGEs increase model of zebrafish. Set 3 parallel experiments. After 24 h treatment on a 60° C. shaker, the supernatant was centrifuged and the data were collected using a multifunctional microplate reader, the fluorescent values of AGEs in each experimental group were analyzed, and the results of statistical analysis of this indicator were used to evaluate the anti-aging efficacy of samples.

TABLE 9
Anti-glycation effect of Dendrobium officinale
(flower) - aqueous extract on zebrafish

| | Concentration | AGEs fluorescence |
|---|---|---|
| Group | (μg/mL) | value (mean ± SE) |
| Blank | — | 34172 ± 1238** |
| Model | — | 40761 ± 485 |
| AG (positive) | 200 | 35106 ± 770** |
| Dendrobium officinale (flower) - | 500 | 38875 ± 575 |
| aqueous extract | 1000 | 33337 ± 344*** |

Compared with Model group,
**p < 0.01,
***p < 0.001

As shown in Table 9 above, Dendrobium officinale (flower)-aqueous extract shows a positive effect for anti-glycation. Thus, it is believed that such positive effect can also be provided to other subjects, including humans.

### Further/Confirmation Studies

In view of the initial studies above, and without being bound or limited to any particular theory, it is believed that D. officinale flowers (DOF) can provide a number of well-being beneficial effects. Therefore, the present study aimed to investigate the in vitro biological potency of DOF aqueous extract and screen its active components. A full summary of the study, and related supplementary material thereof, can be found in the following article: Zhou H, Zhou L, Li B and Yue R (2023) “Anti-cyclooxygenase, anti-glycation, and anti-skin aging effect of Dendrobium officinale flowers' aqueous extract and its phytochemical validation in aging.” Front. Immunol. 14:1095848. The disclosure of this article is incorporated herein by reference in its entirety.

### Methods

Antioxidant tests, including 2,2-diphenyl-1-picrylhydrazyl (DPPH), 2,2′-azino-bis(3-ethylbenzothiazoline-6-sulfonic acid) (ABTS), the ferric reducing ability of plasma (FRAP), and intracellular reactive oxygen species (ROS) level analyses in primary human epidermal keratinocytes, anti-cyclooxygenase2 (COX-2) assay, anti-glycation assay (both fluorescent AGEs formation in a BSA fructose/glucose system and glycation cell assay), and anti-aging assay (quantification of collagen types I and III, and SA-β-gal staining assay) were conducted to determine the potential biological effects of DOF extracts and its major compounds. Ultra-performance liquid chromatography-electrospray ionisation-quadrupole-time-of-flight-mass spectrometry (UPLC-ESI-QTOF-MS/MS) was performed to investigate the composition of DOF extracts. Online antioxidant post-column bioassay tests were applied to rapidly screen major antioxidants in DOF extracts.

### Results and Discussion

The aqueous extract of D. officinale flowers was found to have potential antioxidant capacity, anti-cyclooxygenase2 (COX-2) effect, anti-glycation potency, and anti-aging effects. A total of 34 compounds were identified using UPLC-ESI-QTOF-MS/MS. Online ABTS radical analysis demonstrated that 1-O-caffeoyl-β-D-glucoside, vicenin-2, luteolin-6-C-β-D-xyloside-8-C-β-D-glucoside, quercetin-3-O-sophoroside, rutin, isoquercitrin, and quercetin 3-O-(6″-O-malonyl)-β-D-glucoside are the major potential antioxidants. In addition, all selected 16 compounds exerted significant ABTS radical scavenging ability and effective AGE suppressive activities. However, only certain compounds, such as rutin and isoquercitrin, displayed selective and significant antioxidant abilities, as shown by DPPH and FRAP, as well as potent COX-2 inhibitory capacity, whereas the remaining compounds displayed relatively weak or no effects. This indicates that specific components contributed to different functionalities. The findings justified that DOF and its active compound targeted related enzymes and highlighted their potential application in anti-aging.

### 1. BACKGROUND

The D. officinale flower is popular in China due to its anti-aging properties and beneficial effects on yin deficiency syndromes. However, scientific data to confirm the pharmacological effects listed above is limited in the literature. Therefore, the purpose of this study was to tentatively conduct more systematic experiments on the aqueous extracts of D. officinale, including its biological effects on skin aging, such as antioxidant capacity (through (DPPH), (ABTS), and (FRAP)), anti-inflammatory effect (through COX-2), anti-glycation potency (through inhibition of non-enzymatic glycation reaction and inhibition of CML expression in fibroblasts), and anti-aging evaluation (through the SA-β-gal staining test and collagen expression).

The phytochemical composition of D. officinale was also analyzed using UPLC-ESI-QTOF-MS/MS. Antioxidants in D. officinale flower aqueous extract were identified using the online ultra-performance liquid chromatography-photodiode array detection-mass spectrometry-2,2′-azino-bis(3-ethylbenzothiazoline-6-sulfonic acid (UPLC-PDA-MS-ABTS+·) method and the biological capacities of specific isolated constituents were explored (through DPPH, ABTS, FRAP, anti-COX-2, and AGE inhibitory activities).

Previous research has shown that D. officinale flowers can alleviate brain aging and improve spatial learning abilities in senescent rats. However, previous literature contains few reports on the anti-glycation effect of D. officinale flower aqueous extract. Online antioxidant investigation of D. officinale flower aqueous extract and various biological activities of its identified compounds that are described in the work have not been reported previously.

### 2. MATERIALS AND METHODS

### 2.1 Plant Material, Solvents, and Chemicals

The flowers of D. officinale were purchased by Amway (Shanghai) Technology Co. and authenticated as D. officinale Kimura et Migo flowers by Amway (China) Botanical Research Centre. Analytical-grade methanol was purchased from Honeywell Co. (Charlotte, USA). Acetic acid and liquid LS-MS-HPLC-grade acetonitrile was purchased from Merck (Darmstadt, Germany). Bovine serum albumin (BSA) lyophilised powder, ABTS (98%), DPPH. (98%), 6-hydroxyl-2,5,7,8-tetra-methylchroman-2-carboxylic acid (Trolox), tyrosinase, ascorbic acid, NS-398 (COX-2 inhibitor, CAS: 123653-11-2), and dimethyl sulfoxide (DMSO) were purchased from Sigma-Aldrich (Darmstadt, Germany). High-glucose Dulbecco's modified Eagle's medium (DMEM), fetal bovine serum (FBS), penicillin-streptomycin stock solution (PS, 10,000 U/ml), phosphate-buffered saline (PBS), and pancreatin solution were purchased from Gibco (Carlsbad, CA, USA). The reference standards vicenin-2, luteolin 6-C-f$-D-xyloside-8-C-β-D-glucoside, vicenin-1, luteolin-6-C-β-D-glucoside-8-C-β-D-xyloside, quercetin-3-O-sophoroside, schaftoside, luteolin-6-C-βi-D-glucopyranoside, vicenin-3, apigenin-6-C-βi-D-glucoside-8-C-α-arabinoside, rutin, isoquercitrin, apigenin-6-C-α-L-arabinoside-8-C-βi-D-xyloside, quercetin 3-O-(6″-O-malonyl)-βi-D-glucoside, kaempferol-3-O-rutinoside, astragaline, and isorhamnetin-3-O-glucoside were purchased from Nature Standard Biotech Co. (Shanghai, China). Aminoguanidine hydrochloride (AG) and glucose were purchased from Aladdin (Shanghai, China), and methylglyoxal (MGO) was purchased from Adamas (Delaware, USA). The Cell Counting Kit-8 (CCK-8), FRAP kit, and SA-β-gal staining kit were purchased from Beyotime Biotechnology Co. (Shanghai, China). Alexa Fluor®488 Donkey anti-mouse IgG was purchased from Thermo Fisher (Waltham, Massachusetts, USA). Carboxymethyl lysine antibody, Alexa Fluor® 488 donkey anti-mouse IgG, Alexa Fluor® 488 donkey anti-rabbit IgG, Alexa Fluor© 568 goat anti-rabbit IgG, type I collagen primary antibody, type III collagen primary antibody, and mounting medium with 4′, 6-diamidino-2-phenylindole (DAPI) were purchased from Abcam (Cambridge, UK). The types I and III collagen enzyme-linked immunosorbent assay (ELISA) kits were purchased from BIO-SWAMP Co. (Wuhan, China). The human dermal fibroblasts (HDFs) were purchased from Archgene Biotechnology Co. (Shanghai, China). Zebrafish were purchased from Hunter Biotechnology Co. (Hangzhou, China). All other chemicals were purchased from Titan Co. (Shanghai, China). Water was purified using a Milli-Q purification system (Barnstead, USA).

### 2.2 Extraction Procedure

Aqueous extracts of D. officinale flower (DOF) were prepared using slightly modified methods. Briefly, the pulverised flower powder was reflux-extracted twice with distilled water at a solid-to-solvent ratio of 1:12 (w/v) for 1 h at 100° C. The extract was separated through centrifugation at 19,000×g for 15 min at 4° C. (RWB3220CY-2, Eppendorf, Germany). The supernatant was evaporated at 65° C. using a scale rotary evaporator (Hei-VAP Expert, Heidolph; Schwabach, Germany) until a small volume remained, then lyophilised using a freeze dryer. The DOF hot-water extract (DOF-W) was then stored at −18° C. until further analysis.

### 2.3 Antioxidant Capacity (DPPH-, ABTS·+, and FRAP Assays)

Fast colourimetric methods were slightly modified for the in vitro assessment of DPPH· scavenging, ABTS·+ decolourisation capacity, and total antioxidant capacity of FRAP. The stock solutions of derivatisation reagents were diluted as follows before measurement: DPPH was diluted with absolute ethanol until the absorbance was 0.8±0.05 at λ=517 nm. ABTS working solution was prepared with phosphate buffer (0.2 M, pH 7.4) to display absorbance of 0.8±0.05 at λ=729 nm, based on a previous study. The FRAP solution was prepared according to the instructions of the FRAP kit. Absorbances were measured using an automatic microplate reader (Molecular Group Ltd., USA), and all analyses were performed using 96-well plates. All measurements were performed in triplicate, and Trolox was used as a positive control.

For DPPH· scavenging, 100 μl sample solution was mixed with 100 μl fresh DPPH ethanolic solution, and the absorbance of the mixture was measured after 10 min at 517 nm. Ethanol was used as a negative control.

For ABTS·+ decolourisation capacity, 100 μl of ABTS working solution was mixed with 200 μl of sample solution, and the absorbance was recorded after 10 min at 729 nm. Phosphate buffer (0.2 M, pH 7.4) was used as a negative control.

DPPH· and ABTS·+ radical scavenging activities of the tested sample were both calculated using the following formula:

$$\% inhibition = ( A control - A sample ) / A control \times 100$$

where A control and A sample represent the absorbance of the control and test samples, respectively. The IC₅₀ was calculated graphically from the dose-inhibition curves.

For total antioxidant capacity, 180 μl of FRAP solution was mixed with 5 μl of sample solution, and the absorbance was measured after 5 min at 539 nm with PBS as the negative control. FeSO₄ solutions (0.15, 0.3, 0.6, 0.9, 1.2, and 1.5 mM) were used for the calibration curve. The FRAP value represents the corresponding concentration of FeSO₄ solutions (mM FeSO₄). The regression equation from the standard curve was used to calculate the equivalent concentration 1 (EC₁) of each sample. EC₁ is defined as the concentration of the test sample with an absorbance equivalent to that of 1.0 mmol/L of FeSO₄ solution, determined by its calibration curve.

### 2.4 Cellular Antioxidant Activity Assay

### 2.4.1 Cell Culture and UVB Treatment

Primary human epidermal keratinocytes (NHEKs) were purchased from Lifeline® Cell Technology (Frederick, MD, USA) and cultured in DermaLife K Keratinocyte Calcium-Free Medium (Cat. LL-0029). NHEKs were incubated in a humidified incubator with 5% CO₂ at 37° C. NHEKs were exposed to a spectral peak at 312 nm of the UVB irradiation by using an UVB lamp (Spectroline Model EB-160C, New York, NY, USA) at doses of 10 mJ/cm². After UVB irradiation, the cells were washed with warm PBS, and then fresh medium with and without different concentrations of DOF-W (10 and 40 μg/ml) was added and incubated for 24 h.

### 2.4.2 Cell Viability Assay and Measurement of ROS Generation

Cell viability was determined using the CCK-8. After 24 h of incubation, the optical density (OD) was recorded using a microplate reader at 450 nm, according to the instructions of the kit. Based on the methods reported in a previous study, the relative levels of ROS were detected using CellROX® Reagent (Life Technologies, Waltham, MA USA). Briefly, NHEKs were seeded in 96-well microplates at 2×10⁴ cells/well for 48 h, followed by the UVB treatment and incubation periods specified above. The cells were then incubated with 5 μM CellROX® for 1 h and washed thrice with PBS. ROS production was measured through the fluorescent intensity with the excitation and emission wavelengths set at 485 and 520 nm, respectively.

### 2.5 Anti-COX-2 Assay

Anti-inflammatory activity was assessed by measuring COX-2 enzymatic inhibition on the basis of a described method by using the COX-2 Inhibitor Screening Kit (Beyotime, No. S0168). Celecoxib, a COX-2 inhibitor, was used as a positive control. The results of anti-COX-2 activities are presented as IC₅₀ values (μg/ml or μM), a measurement of the inhibition of enzyme activity by each sample by 50%.

### 2.6 Anti-Glycation Capacity

### 2.6.1 Fluorescent AGEs Formation in a BSA-Fructose/Glucose System

The formation of total fluorescent AGEs in glycated samples was assessed by determining their fluorescent intensities at an excitation/emission wavelength of 350/450 nm, as previously described. Glycated protein was prepared in vitro by incubating BSA in the presence of D-glucose (GLC) and D-fructose (FRC). The DOF extract and specific standards were dissolved in DMSO:water (1:4). BSA (4 mg/ml, 100 μl) was preliminary mixed with 50 μl of GLC (0.5 M) and 50 μl of FRC (0.5 M) in 20 mM sodium phosphate buffer (pH 7.4), and then 100 μl samples were added in the 96-well plate and incubated at 37.5° C. for 7 days. All solutions were prepared under sterile conditions and filtered using a 0.22-μm syringe before incubation. The assay was performed in triplicate, and AG was used as a positive control. The percentage inhibition of fluorescent AGE formation was calculated using the following equation: Inhibition (%)=1−[(IF sample−IF sample control)/(IF control−IF blank control)]×100% where IF sample is fluorescence intensity in the presence of samples and BSA, IF sample control is fluorescence intensity in the presence of samples without BSA, IF control is fluorescence intensity without samples, and IF blank control is fluorescence intensity without samples and BSA. The results of anti-glycation activities are presented as IC₅₀ values (μg/ml or μM), a measurement of the 50% inhibition of enzyme activity by each sample.

### 2.6.2 Glycation Cell Assay

Based on previously reported methods, human primary dermal fibroblasts (HDFs) were induced by MGO to establish a cell model of high CML expression.

HDFs were cultured in high-glucose DMEM supplemented with 10% FBS containing 100 g/ml of PS at 37° C. in a humidified 5% CO₂ incubator. Cell viability was assessed using the CCK-8 kit. HDFs were cultured in 12-well plates to approximately 70% confluence and then treated with 0.5 mmol/L of MGO for 48 h to induce glycation and the formation of CML. Subsequently, different concentrations of samples were added and co-cultured with MGO for an additional 48 h. Cells were then washed once with PBS and fixed in polyformaldehyde overnight at 4° C. Triton-X-100 solution (0.1%) was assigned to fixed cells for 5 min at room temperature to improve cell membrane permeability. After washing twice with PBS, cells were blocked in 1% BSA for 1 h, followed by incubation with the primary CML antibody (1:50) overnight at 4° C. The secondary antibody donkey-anti-Mouse Alexa Fluor® 488 (1:1,000) was then added for 2 h at room temperature. Counterstaining was performed by adopting anti-fluorescence quenching and sealing liquid containing DAPI, followed by visualisation and fluorescence image capture using a microscope (Leica, Germany). ImageJ software was used to quantify CML expression from different images randomly selected in each group.

### 2.7 Anti-Aging Assay on HDFs

### 2.7.1 Cell Culture

HDFs were cultured in DMEM with 10% (v/v) FBS and 1% PS at 37° C. in a humidified 5% CO₂ incubator. Cells were subcultured using pancreatin solution after reaching confluence. All experiments were performed between the third and eighth passages.

### 2.7.2 Quantification of Collagen Types I and III Using Immunofluorescence

The synthesis of collagen types I and III was detected using IF and ELISA. HDFs were digested and seeded in a 24-well culture plate on slides at a density of 50,000 cells/well in a complete medium. After reaching 80% confluency, cells were starved with the medium in the absence of serum for 16 h. Cells were then treated with positive controls or active samples (prepared in medium without serum) for an additional 48 h. For analysis and visualisation of types I and III collagens, the supernatants and remaining cell slides were both collected and analysed using ELISA kits and the IF staining method, respectively.

Collected supernatants were centrifuged at 10,000 rpm for 10 min and used for types I and III collagen ELISA, according to the manufacturer's protocol. The remaining cell slides were washed once with PBS and fixed in polyformaldehyde overnight at 4° C. Subsequently, the slides were treated with 0.5% Triton-X100 for 5 min. After washing, cells were blocked with 1% BSA at room temperature for 1 h. Cells were then incubated with types I and type III collagen primary antibodies (1:100) for 2 h at room temperature. After washing, cells were stained with secondary antibody (1:1,000) for 1 h (Alexa Fluor® 568 goat anti-rabbit IgG and Alexa Fluor® 488 donkey anti-rabbit IgG were used to detecting fluorescence of types I and type III collagen, respectively). Finally, the nuclei were counter-stained with DAPI, and relevant fluorescent images were captured using a microscope. ImageJ software was used to quantify collagen expression from different images randomly selected in each group.

2.7.3 SA-β-gal staining assay

Senescent human skin fibroblasts induced by D-galactose (D-gal) were established based on a previously reported method. HDFs were divided into the control, aging model, and sample (aging model with DOF extract) groups. Except for the control group, HDFs were cultured in six-well plates to approximately 70% confluence and then treated with 20 mg/ml D-gal for 72 h to induce cell senescence. Subsequently, different concentrations of DOF extract were added to the sample group and co-cultured with D-gal for an additional 72 h. The media was then aspirated from cells, and the wells were washed once with PBS. SA-β-gal staining was performed using an SA-β-gal staining kit (Beyotime Co.). Fixative was added and incubated for 10 min at room temperature. Cells were then washed twice with PBS and incubated in 1 ml of SA-β-gal staining solution (freshly prepared according to the protocol of the kit). Plates were maintained in the dark, overnight, in a humidified incubator at 37° C. without CO₂. The following day, the staining solution was removed and cells were maintained in the final solution of PBS. The staining of SA-β-gal was observed, and relevant images were captured using a microscope. ImageJ software was used to determine the average value of the proportion of blue-stained cells of five randomly selected microscopic images in each group.

### 2.8 UPLC-PDA-QTOF-ESI-MS/MS Analysis

DOF extract (1 mg) was diluted with 1 ml of distilled water, sonicated for 20 min, and filtered using a 0.45-μm syringe filter before analysis.

The UPLC analysis was performed using an Agilent 1290 UPLC system (California, USA) combined with an Agilent Q-TOF 6545 LC/MS system, a sample manager, a PDA detector, and a binary solvent manager, and was controlled using MassHunter Workstation Software. The Acquity HSS T3 reverse phase column (2.1×100 mm, 1.8 μm; Waters, Milford, MA, USA) at a separation temperature of 30° C. was used to perform the chromatographic separation of 2 μl of each sample with a wavelength scanning range of 190-400 nm. Gradient elution at a flow rate of 0.2 ml/min was completed with the mobile phase consisting of solvent A (0.2% acetic acid in ultrapure water) and solvent B (acetonitrile) in the following order: 0-5 min, 2% B; 5-8 min, 2%-10% B; 8-12 min, 10% B; 12-20 min, 10%-15% B; 20-28 min, 15%-20% B; 28-31 min, 20% B; 31-38 min, 20-80% B; and 38-40 min, 80% B. Finally, the initial conditions were reintroduced over the course of 2 min. Before each run, the column was equilibrated for an additional 2 min. The MS was operated in both positive and negative ion modes. The optimised MS conditions were as follows: TOF mass range, m/z 50-1,700; ion source gas, 50 psi; curtain gas, 35 psi; ion spray voltage, 5 kV; ion source temperature, 500° C.; and collision energy, 10 eV. The following MS/MS parameters were applied: MS/MS mass range, 50-1,250 m/z; collision energy, 40 eV; declustering potential, 100 V; and collision energy spread, 20 eV. Compounds were identified and analysed by comparing their retention times, fragment ions, and formulas using corresponding standards and commercial databases.

2.9 Antioxidant Online Profiling Using UPLC-PDA-QDa Coupled with Postcolumn Derivatisation with ABTS

Online identification of antioxidant components of DOF extract was performed using a UPLC system (Milford, MA, USA) consisting of a Waters photodiode array detector and a Waters postcolumn derivatisation system supplying fresh ABTS solution (UPLC-PDA-QDa-ABTS). Gradient elution at a flow rate of 0.8 ml/min was completed with the mobile phase consisting of solvent A (0.2% acetic acid in ultrapure water) and solvent B (acetonitrile) in the following order: 0-5 min, 2% B; 5-8 min, 2%-10% B; 8-12 min, 10% B; 12-20 min, 10%-15% B; 20-28 min, 15%-20% B; 28-31 min, 20% B; 31-38 min, 20%-80% B; and 38-40 min, 80% B. Separation of compounds was performed at 30° C. using an Acquity reversed-phase column (4.6×250 mm, 5 μm; Waters, Milford, MA, USA). The detection wavelengths were set at 280 and 734 nm, and the injection volume of the sample was 10 μl.

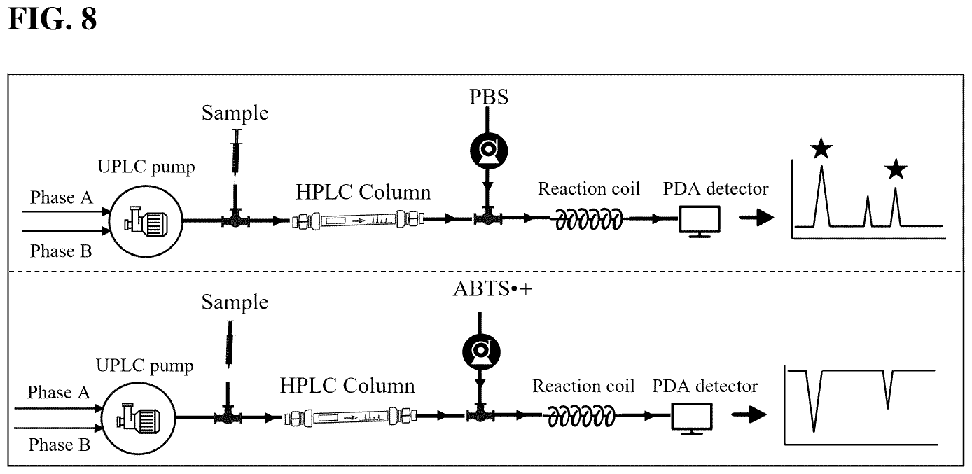

The technical route and relevant device installation are shown in FIG. 8. FIG. 8 is a flow chart of online detection of antioxidants.

To detect radical scavengers, the UPLC system was coupled with a Waters pump, which supplied freshly prepared ABTS·+ solution into a reaction coil (15 m, 0.25 mm ID) with a flow rate of 0.2 ml/min at 37° C. Negative peaks were recorded based on a decrease of absorbance at 729 nm after the reaction of individual compounds with the ABTS·+ radical. For preliminary identification of compounds, a Waters Acquity QDa mass detector in negative ionisation mode was connected in series to PDA with the following parameters: electrospray ion source; cone voltage, 15 V; atomiser, N2; and scanning range, 100-800 m/z.

### 2.10 Statistical Analysis

The data were statistically processed using GraphPad software. The test data were expressed as mean±standard deviation (SD), and significance was evaluated by one-way analysis of variance (ANOVA) and Tukey's test. Statistical significance was indicated as follows: *p<0.05 and **p<0.001.

### 3. RESULTS

### 3.1 In Vitro Antioxidant Potency of DOF Extracts

The growing awareness of herbs with antioxidant properties has been noted over the last few decades, mainly due to the discovery of ROS closely involved in chronic non-infectious diseases. Using several antioxidation assays and various models is vital for a more comprehensive assessment of natural products. Therefore, in the present study, DPPH, ABTS, and FRAP assays, as well as intracellular ROS levels induced by UVB radiation in NHEKs cells, were used for measuring the antioxidant potential of the D. officinale flower aqueous extract (DOF-W).

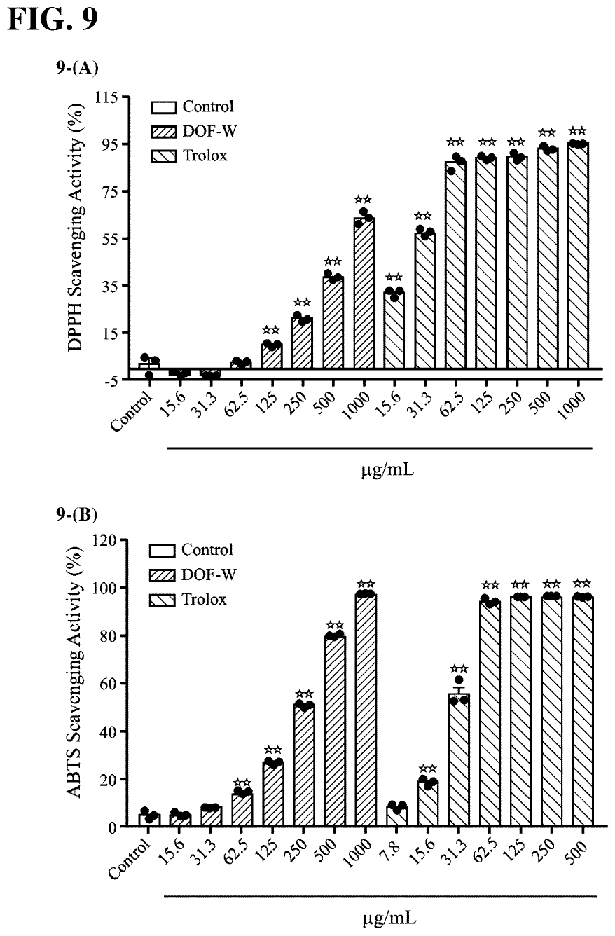

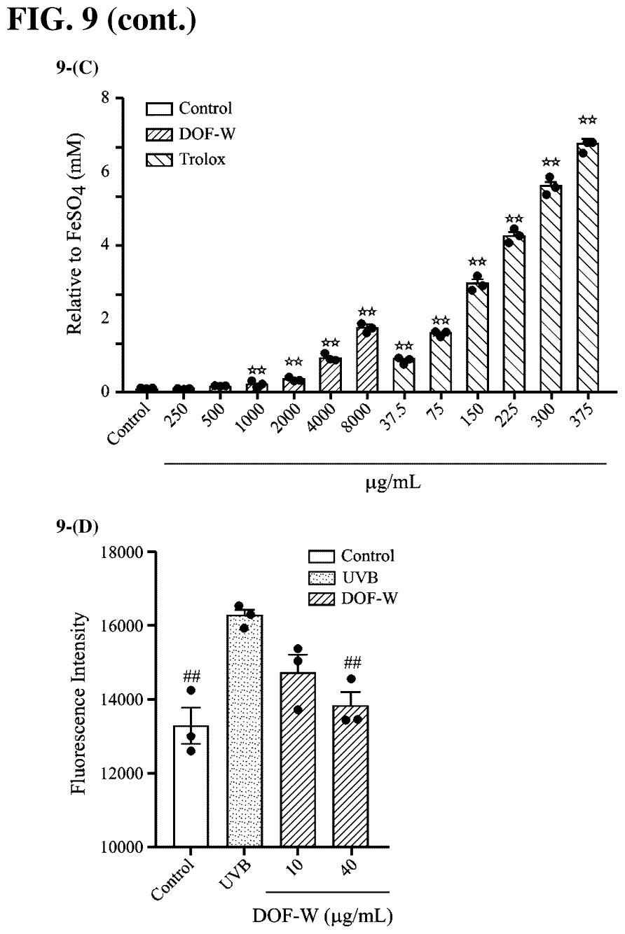

For the concentration range of 15.6-1,000 μg/ml, DOF-W showed antioxidant activity in a dosage-dependent manner, as shown in FIG. 9. Specifically, FIG. 9 shows: antioxidant potency of DOF extracts; specifically: DPPH scavenging activity (A), ABTS scavenging activity (B), FRAP value (C), and intracellular ROS level (D) of DOF-W. As for FIG. 9: compared with the control, **p<0.01; and compared with the UVB model, ^{##}p<0.01.

For the DPPH and ABTS assays, the IC₅₀ value of DOF-W was 669.7±20.59 μg/ml and 224.57±0.65 μg/ml, respectively, whereas the IC₅₀ values of Trolox were 24 7±1.0 μg/ml and 27.68±1.04 μg/ml. For the FRAP assay, the EC1 value of DOF-W was 4,580±260 μg/ml, whereas the EC1 value of Trolox was 55.35±5.22 μg/ml. Though DOF-W showed a weaker antioxidant ability compared to a classical antioxidant standard (Trolox), it still exhibited potential antioxidant activity, particularly at high concentrations, which were consistent with various previous Studies.

Safety and non-toxicity are essential for functional foods, as well as skin-care cosmetics. The DOF-W had no significant damage to NHEK cell viability in the range of 1-40 μg/ml. After being irradiated by UVB (10 mJ/cm²), cell survival was significantly reduced. However, DOF-W did not further aggravate UVB-induced NHEK cell mortality. As FIG. 9-(D) shows, UVB irradiation leads to an increasing ROS level of NHEK cells. Treatment with 40 μg/ml of DOF-W notably reduced intracellular ROS levels.

### 3.2 Anti-COX-2 Capacity of DOF Extracts

As COX-2 is an inducible enzyme that produces prostaglandins (PGs) and is responsible for generating ROS, it is always regarded as a pathologic enzyme chiefly responsible for inflammation, and COX-2 inhibitors, which can cause a sharp drop in the amount of ROS, are also found to be highly associated with potential antioxidant effects. In this study, the COX-2 inhibition assay was used to evaluate the anti-inflammatory effect of DOF-W.

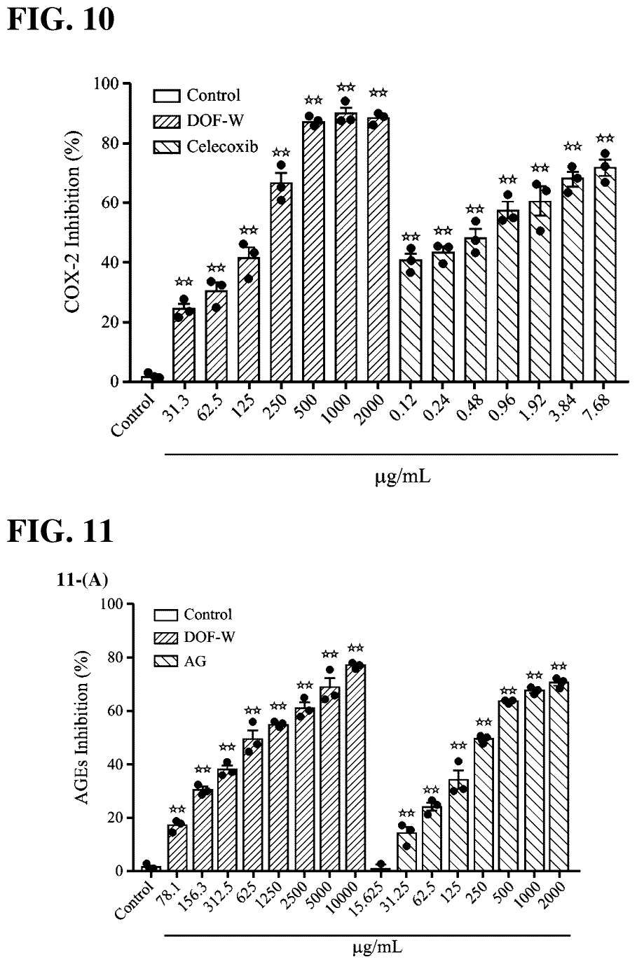

The COX-2 inhibitory activity of DOF-W was compared with a well-known selective COX-2 inhibitor (Celecoxib). For the concentration range of 31.3-2,000 μg/ml, DOF-W showed great concentration-dependent COX-2 inhibitory activity, as shown in FIG. 10 (compared with the control, **p<0.01). The DOF-W had an IC₅₀ value of 133.5±27.4 μg/ml, whereas Celecoxib had an IC₅₀ value of 0.53±0.30 μg/ml. Although DOF-W may show lower capacities due to the dilution of the active constituents with neutral ones, it still could be used as a promising COX-2 inhibitor.

### 3.3 Anti-Glycation Activity of DOF-W

Glycosylation within tissues is a slow and complicated process. Excessive generation and accumulation of AGEs in the process will cause irreversible damage to the body. Various AGEs in skin collagen, including CML, can damage skin fibroblasts, resulting in skin aging and the formation of dark spots. In the present study, the determination of anti-glycation activities was evaluated by multimodal methods, including routine chemical tests, cell experiments, and a zebrafish assay.

### 3.3.1 Inhibition of DOF-W on Total Fluorescent AGEs of Non-Enzymatic Glycation

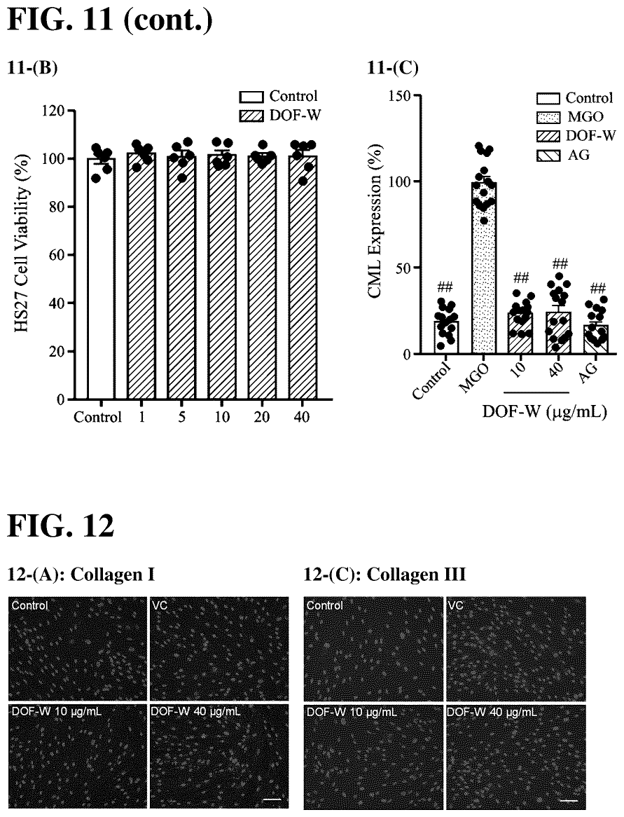

Incubation of reducing sugars and BSA induced the production of fluorescent AGEs. In this study, the BSA-fructose/glucose as a model reaction system was first applied to preliminarily evaluate the effect of DOF aqueous extracts on the inhibition of AGEs. As shown in FIG. 11-(A), DOF-W extracts can reduce levels of fluorescent AGEs in a concentration-dependent relationship with IC₅₀ values of 428.4±75.1 μg/ml. AG, a synthetic AGE inhibitor, expectedly displayed an effective inhibition with IC₅₀ values of 177.3±11.6 μg/ml.

Referring to FIG. 11, it shows: anti-glycation activity of DOF-W. Specifically, FIG. 11 shows: (A) Inhibition of fluorescent AGEs by DOF-W extract, (B) Cytotoxicity of DOF-W extract to fibroblasts, and (C) Effect of CML expression in glyoxal-induced HDFs by DOF-W extract. As for FIG. 11, ^{##}p<0.01 compared with the MGO model. **p<0.01 compared with control. AG, aminoguanidine hydrochloride; MGO, methylglyoxal; CML, N-carboxymethyl lysine. See also FIG. 7, having representative images of HDFs by fluorescence microscope and statistics.

### 3.3.2 Anti-Glycation Effect of DOF-W in Human Primary Fibroblasts

Anti-glycation primary assay of BSA/reducing sugar system as the above showed that DOF extract was a potential anti-glycans. The DOF extract was then further tested in an in vitro glycation assay to confirm its activities on cells. MGO is a critical and potent precursor in the formation of AGEs, reacting with proteins to produce NE-carboxymethyl lysine (CML), which is one of the principal AGEs in the skin without fluorescence properties and cannot be detected by conventional assay. Fibroblasts were treated with MGO to induce glycation, which was then visualised and quantified using IF. No obvious CML green fluorescence staining in the blank control group was observed, which indicated that normal cells hardly secrete CML, whereas a large amount of CML was expressed in MGO-induced cells after CML IF (as previously shown in FIG. 7). The treatment of the cells with the positive compound AG resulted in significant inhibition of glycation by 88.11%. Since no cytotoxicity was found under concentrations of 40 μg/ml, as shown in FIG. 11-(B), treatment with 10 and 40 μg/ml of DOF extract in HDFs both resulted in significant inhibition of glycation by 77.88% and 69.70%, respectively, without a distinct dose-dependent manner (see FIG. 11-(C) and FIG. 7).

### 3.4 Effect of DOF Extracts on Collagen Synthesis in Human Skin Fibroblast Cells

Dermal fibroblasts are thought to be responsible for synthesizing various dermal ECM proteins, including fibrous collagens. Type I collagen accounted for approximately 80% of total collagen, whereas type III collagen is more prevalent in young skin than aged skin and is particularly involved in wound healing. Since skin aging is characterized by the degradation of ECM components such as types I and III collagen breakdown, we investigated whether DOF could enhance the expression of types I and III collagen in HDFs.

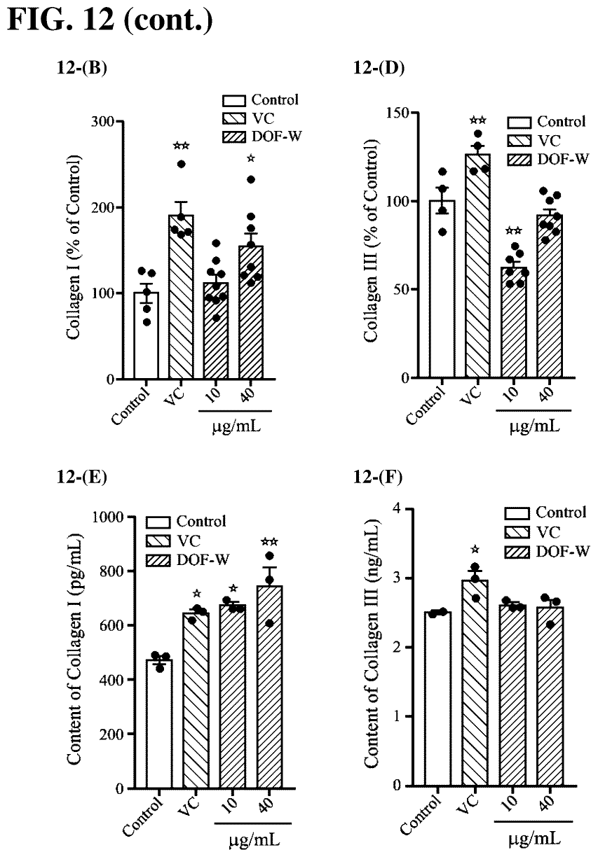

As shown in FIGS. 12-(A) and 12-(B), IF assay revealed that DOF-treated cells synthesized more amount of type I collagen than untreated control cells after 48 h. DOF increased the extent of collagen type I staining by 111.88% and 154.59% of control at a concentration of 10 and 40 μg/ml, respectively. However, the level of expression of type III collagen was not statistically altered or even decreased by DOF extract, as shown in FIGS. 12-(C) and 12-(D). Collagen types I and III expressions were significantly increased by vitamin C by 190.20% and 125.90% of control as a positive drug, respectively.

Referring to FIG. 12, it shows effects of DOF extract on the expression of collagens by IF staining. HDFs were treated with VC (200 μg/ml, positive control) and DOF extract (10 and 40 μg/ml), whereas HDFs were incubated without any treatment as the control group. (A) Images of collagen I staining (red color) and quantification (B). (C) Images of collagen III staining (green color) and quantification (D). Extracellular content of types I (E) and III (F) collagen on HDFs using ELISA. As for FIG. 12: Bar scale, 100 μm; compared with the control group, *p<0.05, **p<0.01.

Also tested was the extracellular content of collagen types I and III for 48 h on HDFs in the culture medium. As shown in FIGS. 12-(E) and 12-(F), treatment with vitamin C (VC), 10 μg/ml of DOF, and 40 μg/ml of DOF increased secreted collagen type I levels by 21.94%, 42.6%, and 57.65%, respectively, whereas DOF had no obvious effect on type III collagen secretion, further confirming the corresponding significantly stronger effect on collagen type I. Therefore, one can conclude that DOF extracts can upregulate collagen type I level in HDFs, but they have no positive effect on type III collagen.

### 3.5 SA-β-Gal Staining Assay on DOF-W

Cellular senescence is an irreversible physiological phenomenon in which normal cells have lost their proliferative potential but are still alive and maintaining their metabolic activity. Cell senescence can be measured using SA-β-gal staining assay, which is widely used to locate SA-β-gal-positive cells (blue-stained cells) by optical microscope. Among several existing types of aging models, such as X-ray, H₂O₂, and D-Gal-induced aging models, D-gal induced aging models to resemble natural aging but take a shorter time than the latter.

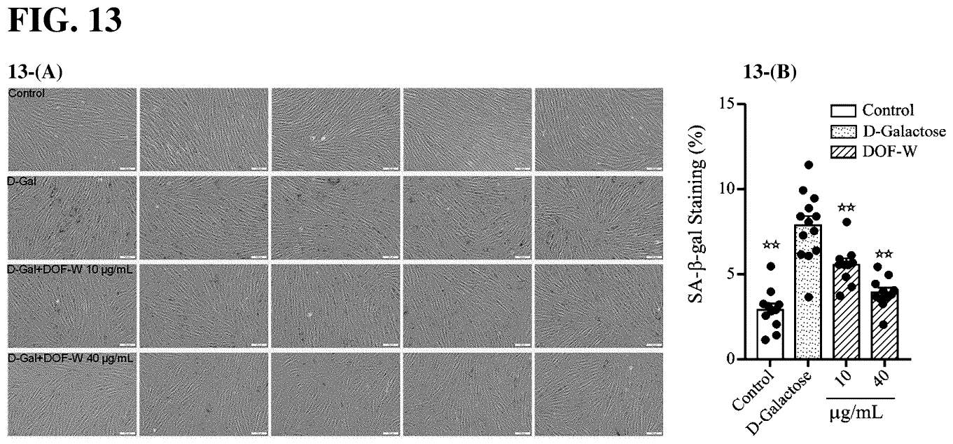

As shown in FIG. 13, few blue-stained cells were observed in the control group (3.0%±1.2%). SA-β-gal-positive cells significantly increased after treatment with 20 mg/ml of D-gal for 6 days, consecutively, in the model group (7.9%±2.0%). When senescent cells coincubated with 10 and 40 μg/ml of DOF extracts, the proportion of SA-j-gal-positive cells were 4.0%±0.9% and 5.6%±1.2%, respectively, which were conspicuously lower than those of the model group.

Referring to FIG. 13, it shows images of fibroblasts with SA-3-gal staining. Statistics results of fibroblasts with SA-3-gal staining. As for FIG. 13: Bar scale, 100 μm. Compared with the model group, **p<0.01.

### 3.6 Determination of Phytochemicals Using UPLC-Q/TOF-MS/MS

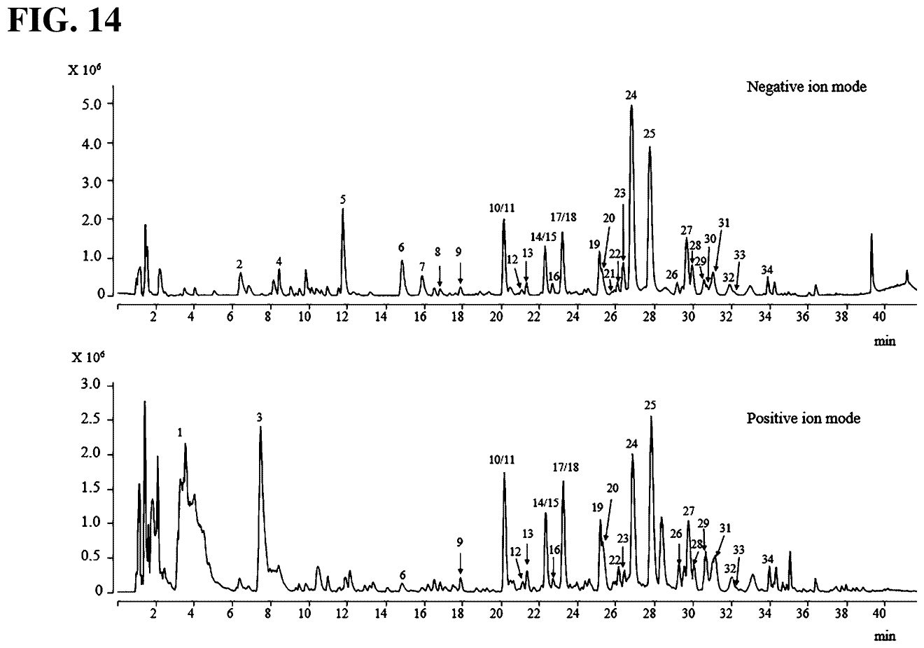

The 25 compounds were further characterised by structural analysis using UPLC-PDA-ESI-Q/TOF-MS/MS. The total ion chromatogram of the aqueous extract of the sample under both negative and positive ion mode is shown in FIG. 14. Specifically, FIG. 14 shows UPLC-ESI/MS/MS total ion chromatogram of DOF-W.

The retention time (Rt), molecular formula, and ion and ions after fragmentation are shown in Table 10. The Rt and fragmentation information of compounds 13, 14, 16, 17, 19, 20, 23, 24, and 25 were compared with those of standards. The major constituents of DOF-W were flavonoids (see Structures 1 below).

TABLE 10
physical properties of the identified compounds from the flowers of D. officinale.

| | | | | | | Mol. |
|---|---|---|---|---|---|---|
| No. | T(min) | Ion | m/z | ppm | Formula | wt. |
| 1 | 3.08 | [M + H]⁺ | 294.1547 | −2.0 | C₁₂H₂₃NO₇ | 293.15 |
| 2 | 5.78 | [M − H]⁻ | 282.0842 | 1.3 | C₁₀H₁₃N₅O₅ | 283.09 |
| 3 | 6.83 | [M + H]⁺ | 268.1047 | 0.5 | C₁₀H₁₃N₅O₄ | 267.10 |
| 4 | 8.04 | [M − H]⁻ | 299.0776 | 3 | C₁₃H₁₆O₈ | 300.08 |
| 5 | 11.49 | [M − H]⁻ | 341.0889 | 4.8 | C₁₅H₁₈O₉ | 342.10 |
| 6 | 14.41 | [M − H]⁻ | 325.0935 | 3.6 | C₁₅H₁₈O₈ | 326.10 |
| 7 | 15.45 | [M − H]⁻ | 325.0935 | 3.6 | C₁₅H₁₈O₈ | 326.10 |
| 8 | 16.53 | [M − H]⁻ | 771.1987 | 0.4 | C₃₃H₄₀O₂₁ | 772.21 |
| 9 | 17.57 | [M − H]⁻ | 609.1461 | 0.2 | C₂₇H₃₀O₁₆ | 610.15 |
| 10 | 19.86 | [M − H]⁻ | 593.1519 | 2.1 | C₂₇H₃₀O₁₅ | 594.16 |
| 11 | 19.86 | [M − H]⁻ | 579.1352 | 1.3 | C₂₆H₂₈O₁₅ | 580.14 |
| 12 | 20.82 | [M − H]⁻ | 579.1355 | −0.2 | C₂₆H₂₈O₁₅ | 580.14 |
| 13 | 21.06 | [M − H]⁻ | 623.162 | 0.0 | C₂₈H₃₂O₁₆ | 624.16 |
| 14 | 22.03 | [M − H]⁻ | 563.1398 | −0.5 | C₂₆H₂₈O₁₄ | 564.15 |
| 15 | 22.03 | [M − H]⁻ | 579.1353 | −0.2 | C₂₆H₂₈O₁₅ | 580.14 |
| 16 | 22.36 | [M − H]⁻ | 625.1396 | −1.4 | C₂₇H₃₀O₁₇ | 626.15 |
| 17 | 22.93 | [M − H]⁻ | 563.1404 | 0.6 | C₂₆H₂₈O₁₄ | 564.15 |
| 18 | 23.07 | [M − H]⁻ | 447.0932 | −0.2 | C₂₁H₂₀O₁₁ | 448.10 |
| 19 | 24.91 | [M − H]⁻ | 563.1426 | 4.5 | C₂₆H₂₈O₁₄ | 564.15 |
| 20 | 25.14 | [M − H]⁻ | 593.1521 | 1.0 | C₂₇H₃₀O₁₅ | 594.16 |
| 21 | 25.56 | [M − H]⁻ | 563.1404 | −0.2 | C₂₆H₂₈O₁₄ | 564.15 |
| 22 | 25.87 | [M − H]⁻ | 533.1309 | 1.1 | C₂₅H₂₆O₁₃ | 534.14 |
| 23 | 26.07 | [M − H]⁻ | 609.1469 | 2.2 | C₂₇H₃₀O₁₆ | 610.15 |
| 24 | 26.52 | [M − H]⁻ | 609.1484 | 4.7 | C₂₇H₃₀O₁₆ | 610.15 |
| 25 | 27.46 | [M − H]⁻ | 463.0897 | 4.4 | C₂₁H₂₀O₁₂ | 464.10 |
| 26 | 29.05 | [M + H]⁺ | 535.1457 | 1.0 | C₂₅H₂₆O₁₃ | 534.14 |
| 27 | 29.38 | [M − H]⁻ | 549.0882 | 0.2 | C₂₄H₂₂O₁₅ | 550.10 |
| 28 | 29.76 | [M − H]⁻ | 593.1517 | 1.7 | C₂₇H₃₀O₁₅ | 594.16 |
| 29 | 30.45 | [M + H]⁺ | 565.1561 | 0.7 | C₂₆H₂₈O₁₄ | 564.15 |
| 30 | 30.82 | [M + H]⁺ | 565.1538 | −3.4 | C₂₆H₂₈O₁₄ | 564.15 |
| 31 | 30.83 | [M − H]⁻ | 447.0942 | 3.3 | C₂₁H₂₀O₁₁ | 448.10 |
| 32 | 31.6 | [M − H]⁻ | 477.1038 | −0.3 | C₂₂H₂₂O₁₂ | 478.11 |
| 33 | 31.86 | [M − H]⁻ | 505.0987 | −0.1 | C₂₃H₂₂O₁₃ | 506.11 |
| 34 | 33.76 | [M − H]⁻ | 533.0935 | −0.3 | C₂₄H₂₂O₁₄ | 534.10 |

| No. | Name | MS/MS data |
|---|---|---|
| 1 | Fructoseleucine | 276.1445; 258.1295; 230.1362; |
| | | 212.1264; 182.1158 |
| 2 | Guanosine | 150.0397; 133.0150; |
| | | 108.0204; 78.9594 |
| 3 | Adenosine | 136.0618; 119.0350; |
| | | 94.0394; 57.0330 |
| 4 | Salicylic acid 2-O-β-D-glucoside | 137.0238; 123.0080; 93.0346 |
| 5 | 1-O-caffeoyl-β-D-glucoside | 179.0347; 161.0246; |
| | | 135.0448; 133.0296 |
| 6 | 1-O-(4-coumaroyl)-β-D-glucose or isomer | 163.0392; 145.0293; |
| | | 117.0345; 59.0135 |
| 7 | 1-O-(4-coumaroyl)-β-D-glucose or isomer | 163.0376; 145.0296; |
| | | 117.0348; 59.0139 |
| 8 | Quercetin 3-O-glucosyl-rutinoside | 771.1974; 609.1456; 463.0794; |
| | | 301.0343; 178.9978 |
| 9 | Luteolin-6-C-β-D-glucoside-8-C-β- | 489.1025; 429.0796; |
| | D-galactoside | 399.0700; 369.0604 |
| 10 | Vicenin-2 | 593.1533; 473.1087; 383.0768; |
| | | 353.0659; 297.0749 |
| 11 | Luteolin-6-C-β-D-xyloside-8-C-β-D-glucoside | 429.0776; 399.0709; |
| | | 369.0602; 339.0487 |
| 12 | Luteolin-6-C-xyloside-8-C-glucoside isomer | 459.0916; 429.0811; 399.0711; |
| | | 369.0605; 339.0518 |
| 13 | Isorhamnetin-3-O-neohesperidoside | 503.1182; 413.0874; 383.0774; |
| | | 357.0621; 315.0635 |
| 14 | Vicenin-1 | 563.1490; 503.1179; 473.1081; |
| | | 383.0680; 353.0667 |
| 15 | Luteolin-6-C-β-D-glucoside-8-C-β-D-xyloside | 429.0799; 399.0710; 369.0616 |
| 16 | Quercetin-3-O-sophoroside | 625.1370; 463.0864; 301.0338 |
| 17 | Schaftoside | 563.1398; 503.1182; 473.1082; |
| | | 383.0770; 353.0664 |
| 18 | Luteolin-6-C-β-D-glucoside | 411.0716; 357.0613; 327.0495; |
| | | 298.0475; 285.0412 |
| 19 | Vicenin-3 | 563.1391; 473.1082; 443.0974; |
| | | 383.0768; 353.0663 |
| 20 | Glucosyl-vitexin | 431.0946; 311.0549; |
| | | 293.0463; 59.0139 |
| 21 | Neoschaftoside | 473.1073; 444.1005; 413.0873; |
| | | 383.0770; 353.0649; 311.0548 |
| 22 | Apigenin-6-C-β-D-xyloside-8-C- | 413.0855; 383.0770; 353.0660 |
| | α-L-arabinoside | |
| 23 | Quercetin-7- | 609.1433; 463.0865; 301.0333; |
| | O-rutinoside | 300.0277; 271.0242 |
| 24 | Rutin | 609.1455; 301.0346; |
| | | 300.0283; 178.9980 |
| 25 | Isoquercitrin | 301.0281; 300.0281; 271.0254; |
| | | 255.0302; 178.9985 |
| 26 | Apigenin-6-C-α-L-arabinoside-8-C-β- | 463.0998; 433.0909; 403.0807; |
| | D-xyloside | 391.0804; 379.0806; |
| | | 325.0697; 307.0595 |
| 27 | Quercetin 3-O-(6″-malonyl-glucoside) | 549.0832; 505.0937; 463.1016; |
| | | 301.0437; 300.0274; |
| | | 271.0244; 255.0295 |
| 28 | Kaempferol-3-O-rutinoside | 593.1506; 285.0398; 284.0323; |
| | | 255.0293; 151.0030 |
| 29 | Apigenin-6-C-arabinosyl-(1→2)-O-β- | 367.0808; 349.0704; 337.0704; |
| | D-glucoside | 313.0702; 283.0598 |
| 30 | Apigenin-8-C-glucosyl-(1→2)- | 367.0810; 349.0697; 337.0704; |
| | α-L-rabinoside | 313.0702; 283.0601 |
| 31 | Astragalin | 284.0320; 255.0293; |
| | | 227.0343; 151.0034 |
| 32 | Isorhamnetin-3-O-glucoside | 314.0426; 299.0176; 285.0401; |
| | | 271.0237; 257.0448; 243.0291 |
| 33 | Quercetin 3-O-(6″-acetyl-glucoside) | 300.0273; 271.0243; |
| | | 225.0307 |
| 34 | Kaempferol 3-O-(6″-malonylglucoside) | 284.0326; 255.0299; |
| | | 229.051 |

### Structures 1: Chemical Structures of Main Herb Markers in the DOF-W

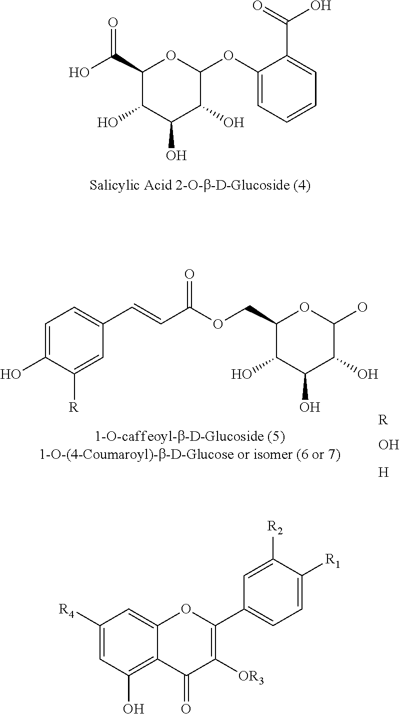

| | R₁ | R₂ | R₃ | R₄ |
|---|---|---|---|---|
| Quercetin 3-O-glucosyl- | OH | OH | glu-rha- | OH |
| rutinoside (8) | | | (1-6)-glu | |
| Isorhamnetin-3-O- | OH | OCH₃ | glu-rha | OH |
| neohesperidoside (13) | | | | |
| Quercetin-3-O-sophoroside (16) | OH | OH | glu-(1-2)- | OH |
| | | | glu | |
| Quercetin-7-O-rutinoside (23) | OH | OH | OH | rha-(1- |
| | | | | 6)-glu |
| Rutin (24) | OH | OH | rha-(1-6)- | OH |
| | | | glu | |
| Isoquercitrin (25) | OH | OH | glu | OH |
| Quercetin-3-O-(6″-malonyl- | OH | OH | malonyl- | OH |
| glucoside) (27) | | | glu | |
| Kaempferol-3-O-rutinoside (28) | OH | H | rha-(1-6)- | OH |
| | | | glu | |
| Astragalin (31) | OH | H | glu | OH |
| Isorhamnetin-3-O-glucoside (32) | OH | OCH₃ | glu | OH |
| Quercetin 3-O-(6″-acetyl- | OH | OH | acetyl-glu | OH |
| glucoside) (33) | | | | |
| Kaempferol 3-O-(6″- | OH | H | malonyl- | OH |
| malonylglucoside) (34) | | | glu | |

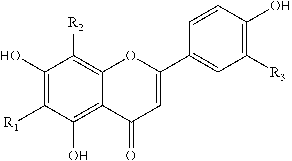

| | R₁ | R₂ | R₃ |
|---|---|---|---|
| Luteolin-6-C-β-D-glucoside -8-C-β-D- | glu | gal | OH |
| galactoside (9) | | | |
| Vicenin-2 (10) | glu | glu | H |
| Luteolin-6-C-xyloside-8-C-glucoside (11 or 12) | xyl | glu | OH |
| Vicenin-1 (14) | xyl | glu | H |
| Luteolin-6-C-glucoside-8-C-xyloside (15) | glu | xyl | OH |
| Schaftoside (17) | glu | α-L- | H |
| | | ara | |
| Luteolin-6-C-β-D-glucoside (18) | glu | H | OH |
| Vicenin-3 (19) | glu | xyl | H |
| Glucosyl-vitexin (20) | H | glu-glu | H |
| Neoschaftoside (21) | glu | β-L- | H |
| | | ara | |
| Apigenin-6-C-β-D-xyloside-8-C-α-L- | xyl | ara | H |
| arabinoside (22) | | | |
| Apigenin-6-C-α-L-arabinoside-8-C-β-D- | ara | xyl | H |
| xyloside (26) | | | |
| Apigenin-6-C-arabinosyl-(1→2)-O-β-D- | ara-(1- | H | H |
| glucoside (29) | 2)-glc | | |
| Apigenin-8-C-glucosyl-(1→2)-α-L- | H | glu-(1- | H |
| arabinoside (30) | | 2)-ara | |

### 3.7 Determination of Antioxidants by the Online UPLC-PDA-MS-ABTS·+ Scavenging

To preliminarily screen out antioxidant phytochemicals in DOF-W, in the present study, UPLC coupled with PDA-Qda and ABTS-based assay was performed. The constituents, for which MS data and reference compounds were available, can be identified from positive peaks. The area of reordered negative peaks on the lower chromatogram at 734 nm conforms to the ABTS radical scavenging activity of individual compounds of the DOF extract. As it may be judged from the size of the negative peaks in the chromatogram, 1-O-caffeoyl-βi-D-glucoside (compound 5), vicenin-2 (compound 10), luteolin-6-C-βi-D-xyloside-8-C-β-D-glucoside (compound 11), quercetin-3-O-sophoroside (compound 16, rutin (compound 24), isoquercitrin (compound 25), and quercetin 3-O-(6″-O-malonyl)-βi-D-glucoside (compound 27) were the strongest ABTS radical scavengers in the investigated extract. Since components 10 and 11 are not well separated, it is a challenge to distinguish which component is mainly responsible for the negative peak and needs further confirmation by extra experiments. In a previous report, by employing precolumn DPPH and ABTS assay followed by HPLC-DAD analysis, 1-O-caffeoyl-β-D-glucoside, rutin, and isoquercitrin were identified as major components with obvious scavenging free radical abilities in methanol extract of DOF.

3.8 In Vitro Pro-Health Potency of Specific Compounds from DOF Extracts

In consideration of the time-consuming process of isolating components in flowers and to further identify and confirm the biological activities of components that may contribute to the antioxidative, anti-inflammatory, and anti-glycation properties of the extract, specific available compounds were selected for more detailed investigation on the assays of DPPH, ABTS, FRAP, COX-2, and BSA/reducing sugar glycation. The results of these isolated compounds are shown in Table 11.

TABLE 11
Antioxidant (DPPH, ABTS, FRAP), anti-cyclooxygenase (COX-2), and AGE inhibition
activity (BSA/reducing sugar reaction) (% of inhibition) of various compounds
from Dendrobium officinale flower-aqueous extract.

| | | | | | | BSA- |
|---|---|---|---|---|---|---|
| | | DPPH | ABTS | FRAP | COX-2 | glycation |
| | | IC₅₀ | IC₅₀ | EC₁ | IC₅₀ | IC₅₀ |
| No. | Compound name | (mM) | (mM) | (mM) | (mM) | (mM) |
| 10 | Vicenin-2 (apigenin 6,8-C- | >5,000 | 74.41 ± | >5,000 | nd | 257.67 ± |
| | diglucoside) | | 0.69 | | | 7.56 |
| 11 | Luteolin 6-C-β-D-xyloside- | 226.93 ± | 15.90 ± | 1,009.1 ± | 14.2 ± | 251.6 ± |
| | 8-C-β-D-glucoside | 47.26 | 0.19 | 143.3 | 3.4 | 5.1 |
| 14 | Vicenin-1 (apigenin 6-C- | >5,000 | 96.39 ± | >5,000 | nd | 255.63 ± |
| | β-D-xyloside-8-C-glucoside) | | 2.11 | | | 5.44 |
| 15 | Luteolin-6-C-β-D- | 145.13 ± | 13.80 ± | 1,100.3 ± | 13.9 ± | 293.6 ± |
| | glucoside-8-C-β-D-xyloside | 51.37 | 0.82 | 138.4 | 2.4 | 13.3 |
| 16 | Quercetin-3-O-sophoroside | 172.13 ± | 6.63 ± | 949.6 ± | 11.0 ± | 295.7 ± |
| | | 76.03 | 0.41 | 41.9 | 0.7 | 8.5 |
| 17 | Schaftoside (apigenin 6-C- | >5,000 | 75.59 ± | >5,000 | nd | 250.30 ± |
| | β-D-xyloside-8-C-arabinoside) | | 12.12 | | | 4.16 |
| 18 | Luteolin-6-C-β-D- | 102.28 ± | 7.10 ± | 506.6 ± | 7.9 ± | 316.6 ± |
| | glucopyranoside | 6.20 | 0.10 | 78.3 | 2.4 | 15.6 |
| 19 | Vicenin-3 (apigenin 6-C- | >5,000 | 74.76 ± | >5,000 | nd | 242.90 ± |
| | β-D-glucoside-8-C-xyloside) | | 0.92 | | | 11.97 |
| 21 | Neoschaftoside | >5,000 | 21.45 ± | >5,000 | nd | 430.3 ± |
| | | | 0.24 | | | 8.0 |
| 24 | Rutin | 43.33 ± | 33.09 ± | 205.0 ± | 10.3 ± | 222.03 ± |
| | | 1.75 | 0.37 | 12.2 | 2.8 | 13.49 |
| 25 | Isoquercitrin | 197.30 ± | 10.67 ± | 520.2 ± | 15.4 ± | 212.7 ± |
| | | 42.07 | 0.03 | 17.9 | 3.7 | 10.4 |
| 26 | Apigenin-6-C-α-L- | >5,000 | 80.36 ± | >5,000 | nd | 272.7 ± |
| | arabinoside-8-C-β-D-xyloside | | 0.77 | | | 4.7 |
| 27 | Quercetin 3-O-(6″-O- | 24.70 ± | 28.36 ± | 826.7 ± | 12.5 ± | 288.2 ± |
| | malonyl)-β-D-glucoside | 0.96 | 0.47 | 98.5 | 2.9 | 17.9 |
| 28 | Kaempferol-3-O-rutinoside | >5,000 | 35.01 ± | >5,000 | nd | 374.4 ± |
| | | | 0.22 | | | 18.1 |
| 31 | Astragaline (kaempferol 3- | >5,000 | 34.31 ± | >5,000 | nd | 275.7 ± |
| | β-D-glucopyranoside) | | 0.10 | | | 33.0 |
| 32 | Isorhamnetin-3-O- | 2,366.67 ± | 44.71 ± | 2,757.3 ± | 535.3 ± | 209.2 ± |
| | glucoside | 87.61 | 1.52 | 103.8 | 150.0 | 3.1 |
| Positive | Trolox | 158.87 ± | 145.67 ± | 559.8 ± | | |
| | | 4.47 | 1.22 | 19.4 | | |
| | Celecoxib | | | | 89.7 ± | |
| | | | | | 6.4 | |
| | Aminoguanidine | | | | | 1,604.1 ± |
| | hydrochloride | | | | | 104.5 |

Note:
nd, not detected; values represented as mean ± standard deviation (n = 3); values in the same columns followed by different letters are significantly different at p ≤ 0.05 according to Tukey's test.

In view of the limitations of different antioxidant methods, the use of at least two or more assays with different mechanisms of oxidation is strongly recommended. Three in vitro assays (DPPH, ABTS, and FRAP) were performed to comprehensively analyse the antioxidant capacities of selected compounds. These examined compounds demonstrated various influences on these functional activities. All compounds were observed to display significant scavenging capacities against the ABTS radical, with IC₅₀ values ranging from 6.63±0.41 μM (compound 16) to 96.39±2.11 μM (compound 14). However, as for the DPPH radical and FRAP assay, some compounds (compounds 10, 14, 17, 19, 21, 26, 28, and 31) showed a low level of inhibition with IC₅₀ values that were even higher than 5,000 μM, whereas other compounds (compounds 11, 15, 16, 18, 24, 25, and 27) exhibited extraordinary inhibition, with IC₅₀ of DPPH varying from 24.70±0.96 to 226.93±47.26 μM and EC₁ of FRAP ranging from 205.0±12.2 to 1,100.3±138.4 μM, respectively. The remaining compound 32 had a relatively weak inhibition, with an IC₅₀ of DPPH of 2,366.67±87.61 and an EC₁ of FRAP of 2,757.3±103.8. Based on previous reports, flavonoids appear to exhibit anti-inflammatory properties through the modulation of ROS. According to the present findings, the present data from the anti-COX-2 assay indeed revealed a similar tendency, which was in line with the results observed in the DPPH radical and FRAP assays. Compounds 11, 15, 16, 18, 24, 25, and 27 showed great COX-2 inhibition, with the lowest IC₅₀ being 7.9±2.4 μM, as opposed to no inhibitory effects of compounds 10, 14, 17, 19, 21, 26, 28 and 31. Similar to the antioxidant results (DPPH and FRAP) of compound 32, its inhibitory effect on COX-2 was weak with an IC₅₀ of 535.3±150.0 μM.

Analysis of the influence on AGE formation illustrated that all compounds were observed to display excellent anti-AGE effects, with IC₅₀ ranging from 209.2±3.1 to 374.4±18.1 μM. All the results are very close to that of rutin (IC₅₀, 222.03±13.49 μM), which is not only the main component of DOF-W extracts but also a well-known AGE inhibitor, and the inhibitory effects of all compounds are even better than aminoguanidine hydrochloride (1,604.1±104.5 μM), another frequently used synthetic AGE inhibitor.

### 4. DISCUSSION

In the present study, the results demonstrated that DOF-W exhibited promising antioxidant capacity (DPPH, ABTS, FRAP, intracellular ROS level in NHEK cells), anti-COX-2 effect, anti-glycation potency (inhibition of non-enzymatic glycation reaction and inhibition of CML expression in fibroblasts), and anti-aging effect (SA-β-gal staining test and collagen expression in fibroblasts). In addition, chemical and cellular anti-glycation activity as well as the anti-COX-2 effect on DOF-W were reported for the first time.

Oxidative stress and inflammation caused by unstable free radicals, which are highly deleterious to cells and skin, are both major contributors to the aging process. Compared with younger skin, elderly skin is more susceptible to environmental stimuli and needs external support such as antioxidants. It is demonstrated that DOF-W had obvious antioxidant and anti-inflammatory effects based on chemical, enzymatic, and cellular methods. Previous studies indicated that DOF-W could increase the antioxidant status and inhibit the inflammatory response in alcohol-impaired mice, which is consistent with the experimental results herein.

Online UPLC-PDA-MS-ABTS·+ scavenging results indicated that seven compounds (5, 10 or 11, 16, 24, 25, and 27) are most likely to be major contributors to the overall antioxidant potential of DOF-W extracts. However, although all selected compounds displayed an excellent capacity to scavenge ABTS radicals, only compounds 11, 15, 16, 18, 21, 24, 25, and 27 showed great antioxidant potency with respect to DPPH and FRAP assays. Due to the absence of standard 5, its authentic antioxidant capacity could not be verified at this time. Combining the results of online and traditional chemical methods, compounds 11, 16, 24, 25, and 27 were predicted to be the main antioxidants of DOF-W extract. Based on previous reports, flavonoids appear to exhibit anti-inflammatory properties via the modulation of ROS. The anti-cyclooxygenase-2 assay was indeed observed to reveal a similar tendency as the results of the DPPH radical and FRAP assays. Only compounds 11, 15, 16, 18, 21, 24, 25, and 27 showed outstanding COX-2 inhibitory effects. According to the present study, the antioxidant and anti-inflammatory effects of DOF-W extract are more likely to be attributed to compounds 11, 15, 16, 18, 24, 25, and 27. Luteolin C-glycosylflavones such as compounds 11, 15, and 18 demonstrated great antioxidant and COX-2 inhibitory effects. On the other hand, though apigenin, as one of the most widely distributed flavonoids in the plant kingdom and most frequently studied by researchers, is characterized as a fantastic free-radical scavenger and a remarkable anti-inflammatory agent, apigenin C-glycosylflavones such as compounds 10, 14, 17, 19, 21, and 26 exhibited very weak biological effects. Among these compounds, compounds 11 and 15 possess the same glycosidic bond as compounds 14 and 19, respectively. Therefore, it is believed that the type of aglycone rather than C- or O-glycosides of flavonoids, had a great effect on antioxidant and COX-2 inhibitory potency. It has also been reported that both luteolin and apigenin C-glycosylflavones had much lower inflammatory effects than those observed with their corresponding aglycones and O-glycosides in LPS-induced RAW264.7. The results herein may be explained by the speculation that C-glycosylation of apigenin leads to a reduction of antioxidant and anti-inflammatory potential. Since most of the flavonoids in plants exist primarily as O-glycosides, C-glycoside flavonoids received relatively less attention than flavonoid O-glycosides, especially in the absence of comprehensive studies on their biological benefits. It is more purposeful to explore the pharmacokinetic properties of flavonoid C-glycosides and their bioactivities.

Numerous studies have also shown that external stimuli such as excessive free radicals and spontaneous AGE generation in vivo, which are irreversible once formed in the body, are associated with skin fibroblast damage, destruction of collagen and elastic fibres, a yellow complexion without splendour, and aging deterioration. DOF-W extract displayed excellent capacity to inhibit AGE formation with low IC₅₀ values and decreased CML expression obviously in MGO-induced fibroblasts. In addition, all selected compounds were observed to have a potent capacity to inhibit AGE formation. Therefore, anti-glycation ability of DOF-W may be justified by the synergistic action of most polyphenolic compounds present in the extract. So far, several studies have reported that various flavonoids, both O- and C-glycosides, can cause a marked decrease on AGE production in several in vitro and in vivo experimental models.

Human skin fibroblasts, which primarily exist in the dermis, can form a large amount of collagen, which is a key factor in maintaining skin elasticity. Studies have reported that skin aging may be related to the accumulation of aging fibroblasts within human skin. Both immunofluorescence (IF) assay and extracellular ELISA assay showed that DOF-W extracts can increase the expression of collagen type I, but they have no obvious effect on collagen type III. Moreover, DOF-W can also significantly decrease the proportion of D-Gal-induced senescent cells. In previous research, it had been reported that the Dendrobium officinale flower can alleviate brain aging and improve spatial learning abilities in senescent rats.

### 5. CONCLUSIONS

Based on the current research regarding in vitro studies and phytochemistry analysis, it is suggested that DOF-W is potent with antioxidation, anti-glycation, and anti-aging effects and deserves further research and development. Therefore, it is believed that both DOF-W and its specific compounds may be promising agents for skin anti-aging.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, the term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A composition for administration to a subject, the composition comprising at least one botanical active component, wherein the botanical active component comprises at least one extract selected from the group consisting of:
i) an extract of Dendrobium officinale flower;
ii) an extract of Scrophularia ningpoensis;
iii) an extract of Eucommia ulmoides; and
iv) combinations of i) to iii);
wherein the botanical active component is present in the composition in an amount effective to inhibit advanced glycation end products (AGEs) in skin of the subject.

2. The composition according to claim 1, wherein the botanical active component comprises the extract of Dendrobium officinale flower, optionally wherein the botanical active component consists of the extract of Dendrobium officinale flower.

3. The composition according to claim 2, wherein the extract of Dendrobium officinale flower is obtained by water extracting (or aqueous extracting) flowers or flower-based plant material of Dendrobium officinale.

4. The composition according to claim 2, wherein the extract of Dendrobium officinale flower is obtained by alcohol extracting, optionally ethanol extracting, flowers or flower-based plant material of Dendrobium officinale.

5. The composition according to claim 1, wherein the composition is substantially to completely free of components obtained from non-flower-based plant material of Dendrobium officinale.

6. The composition according to claim 1, wherein the botanical active component comprises the extract of Scrophularia ningpoensis, optionally wherein the extract of Scrophularia ningpoensis is further defined as an extract of Scrophularia ningpoensis radix.

7. The composition according to claim 1, wherein the botanical active component comprises the extract of Eucommia ulmoides, optionally wherein the extract of Eucommia ulmoides is further defined as an extract of Eucommia ulmoides male flower.

8. The composition according to claim 1, wherein the botanical active component further comprises an extract selected from the group consisting of:
i) an extract of Phyllanthus emblica;
ii) an extract of Sophora japonica;
iii) an extract of Aronia;
iv) an extract of Punica granatum; and
v) combinations of i) to iv).

9. The composition according to claim 8, wherein the botanical active component comprises the extract of Phyllanthus emblica, optionally wherein the extract of Phyllanthus emblica is further defined as an extract of Phyllanthus emblica fruit.

10. The composition according to claim 8, wherein the botanical active component comprises the extract of Sophora japonica, optionally wherein the extract of Sophora japonica is further defined as an extract of Sophora japonica flower.

11. The composition according to claim 8, wherein the botanical active component comprises the extract of Aronia, optionally wherein the extract of Aronia is further defined as an extract of Aronia fruit.

12. The composition according to claim 8, wherein the botanical active component comprises the extract of Punica granatum, optionally wherein the extract of Punica granatum is further defined as an extract of Punica granatum fruit.

13. The composition according to claim 1, wherein the composition is further defined as a topical composition that is formulated for topical administration to the subject.

14. The composition according to claim 13, further comprising a cosmetically acceptable carrier, optionally wherein the cosmetically acceptable carrier is not naturally occurring.

15. The composition according to claim 1, wherein the composition is further defined as an oral composition that is formulated for oral administration to the subject.

16. The composition according to claim 15, further comprising a pharmaceutically acceptable additive, optionally wherein the pharmaceutically acceptable additive is not naturally occurring.

17. The composition according to claim 1:
i) further comprising a cosmetically acceptable carrier, wherein the cosmetically acceptable carrier is not naturally occurring;
ii) further comprising a pharmaceutically acceptable additive, wherein the pharmaceutically acceptable additive is not naturally occurring; or
iii) both i) and ii).

18. A method of inhibiting advanced glycation end products (AGEs) in skin of a subject, the method comprising administering an effective amount of a composition to the subject, wherein the composition is according to claim 1.

19. The method of claim 18, wherein the composition is administered topically to the subject.

20. The method of claim 18, wherein the composition is administered orally to the subject.
